SEQUENCE LISTING (8 sequences)

SEQ ID NO: 1 - DNA, 23 bp, Artificial Sequence
  Synthetic primer
caacagagggagtgtgagaaagc

SEQ ID NO: 2 - DNA, 22 bp, Artificial Sequence
  Synthetic primer
aacctcgccgttcttgtaaagc

SEQ ID NO: 3 - DNA, 22 bp, Artificial Sequence
  Synthetic primer
gaatgaagagattaccgctctg

SEQ ID NO: 4 - DNA, 22 bp, Artificial Sequence
  Synthetic primer
ccaagatcaagtccataactgc

SEQ ID NO: 5 - DNA, 22 bp, Artificial Sequence
  Synthetic primer
tgatgtttgctttgctcgtcac

SEQ ID NO: 6 - DNA, 24 bp, Artificial Sequence
  Synthetic primer
gaactgtagggattgatgtgatgc

SEQ ID NO: 7 - DNA, 21 bp, Artificial Sequence
  Synthetic primer
ggagggcaggactgatctctg

SEQ ID NO: 8 - DNA, 22 bp, Artificial Sequence
  Synthetic primer
gcgtaatataggctacaacacc

(19) United States
(12) Patent Application Publication
(10) Pub. No.: US 2015/0157633 A1
(43) Pub. Date: Jun. 11, 2015

(54) WNT PROTEIN SIGNALLING INHIBITORS

(71) Applicant: The Board of Regents of the University of Texas System, Austin, TX (US)

(72) Inventors: Lawrence Lum, Dallas, TX (US); Michael Roth, Dallas, TX (US); Baozhi Chen, Addison, TX (US); Michael Dodge, Boston, MA (US); Chuo Chen, Dallas, TX (US)

(73) Assignee: The Board of Regents of the University of Texas System, Austin, TX (US)

(21) Appl. No.: 14/400,001

(22) PCT Filed: May 6, 2013
(86) PCT No.: PCT/US13/39655

§ 371 Date: Nov. 10, 2014

Related U.S. Application Data
(60) Provisional application No. 61/645,924, filed on May 11, 2012.

(51) Int. Cl. A61K 31/519; A61K 31/495; A61K 31/42; A61K 31/4709; A61K 45/06; A61K 31/44; A61K 31/4439; A61K 31/453; A61K 31/353; A61K 31/444; A61K 31/517

(52) U.S. Cl. CPC A61K 31/519; A61K 31/444; A61K 31/495; A61K 31/42; A61K 31/4709; A61K 31/517; A61K 31/44; A61K 31/4439; A61K 31/453; A61K 31/353; A61K 45/06

(57) ABSTRACT

The present invention generally relates to protein signalling. In particular, compounds that inhibit the Wnt protein signalling pathway are disclosed. Such compounds may be used in the treatment of Wnt protein signalling-related diseases and conditions such as cancer, degenerative diseases, type II diabetes and osteopetrosis.

The present application claims benefit of priority to U.S. Provisional Application Ser. No. 61/645,924, filed May 11, 2012, the entire contents of which are hereby incorporated by reference.

This invention was made with government support under grant number 5R21HD061303 awarded by the National Institutes of Health. The government has certain rights in the invention.

### BACKGROUND OF THE INVENTION

1. Field of the Invention

The present invention generally relates to the fields of molecular biology and medicine. More particularly, it concerns the discovery of compounds that inhibit Wnt-mediated signal transduction pathways, including the Wnt/β-catenin pathway.

2. Description of Related Art

The evolutionary elaboration of gene families in complex multicellular animals provides diverse instructive cellular cues based on single signaling modalities and safeguards against genetic insults. During development, members of the Wnt family of signaling molecules—nineteen in all—contribute to almost all aspects of vertebrate development through induction of unique and shared cellular responses (Angers and Moon, 2009; van Amerongen and Nusse, 2009). In post-embryonic animals, their functions are essential to homeostatic tissue renewal and regeneration (Reya and Clevers, 2005). Similar to that of several other signal transduction pathways that have been shown to be important to cell fate decision-making, activity of the Wnt/β-catenin pathway maintains transcriptional programs that enable stem cells to retain their multi-potency (Cole et al., 2008; Van der Flier et al., 2007). Inability to sustain these transcription programs, perhaps through loss of members of the TCF/LEF family of transcriptional effectors or the β-catenin transcriptional co-activator, results in compromised ability of stem cells to self-renew (Cole et al., 2008; Fevr et al., 2007; Korinek et al., 1998; Muncan et al., 2007).

Pathological states that may arise from altered stem cell function, such as degenerative diseases and cancer, are frequently associated with changes in Wnt/β-catenin pathway activity. Indeed, hyperactivation of the Wnt/β-catenin pathway is thought to induce premature senescence of stem cells and age-related loss of stem cell function (Brack et al., 2007; Liu et al., 2007). In cancer, hyperactivation of the Wnt/β-catenin pathway, often in conjunction with mutations in other cell growth regulatory genes, can lead to aberrant cell growth (Reya and Clevers, 2005). Notably, 90% of colorectal cancers are initiated by the loss of the adenomatosis polyposis coli (APC) gene, a major suppressor of the Wnt/β-catenin pathway (Kinzler and Vogelstein, 1996; Sjoblom et al., 2006). Less frequently, loss of extracellular inhibitors that normally suppress Wnt protein function may give rise to Wnt ligand-dependent tumors (Polakis, 2007). More recently, Wnt-mediated cellular responses that are not dependent upon β-catenin (so called “non-canonical pathways” have also been shown play important roles in cancer (Veeman et al., 2003).

Accordingly, identification of methods and compounds that modulate the Wnt-dependent cellular responses may offer an avenue for therapeutic treatment of diseases associated with aberrant activity of these pathways.

### SUMMARY OF THE INVENTION

The present invention generally provides compounds and their use as Wnt protein signalling inhibitors. Also provided are methods of synthesis of these compounds and pharmaceutical compositions thereof.

Accordingly, in one aspect, the present invention provides a method of inhibiting Wnt protein signalling in a cell comprising administering to the cell an effective amount of a compound of formula:

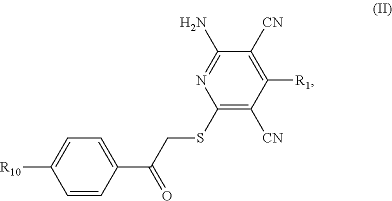

wherein:
   R₁ is alkyl_{(C≦8)} or substituted alkyl_{(C≦8)}; and
   R₁₀ is aryl_{(C≦8)}, substituted aryl_{(C≦8)}, heterocycloalkyl_{(C≦8)} or substituted heterocycloalkyl_{(C≦8)};

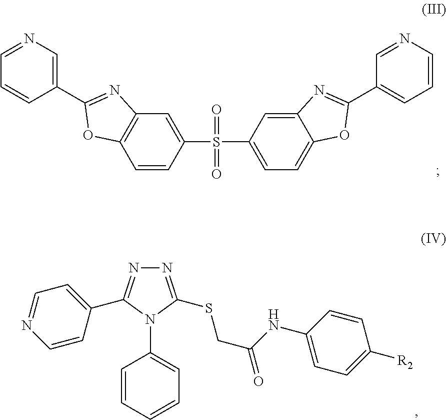

wherein R₂ is alkoxy_{(C≦8)}, substituted alkoxy_{(C≦8)}, acyl_{(C≦8)}, substituted acyl_{(C≦8)}, or heterocycloalkyl_{(C≦8)} or substituted heterocycloalkyl_{(C≦8)};

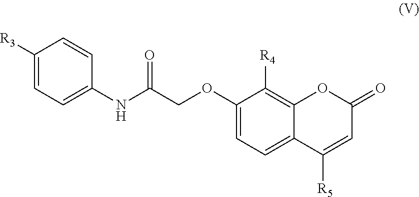

wherein:
   R₃ is acyl_{(C≦8)}, substituted acyl_{(C≦8)}, heterocycloalkyl_{(C≦8)} or substituted heterocycloalkyl_{(C≦8)};
   R₄ is hydrogen, alkyl_{(C≦8)} or substituted alkyl_{(C≦8)}; and
   R₅ alkyl_{(C≦8)} or substituted alkyl_{(C≦8)};

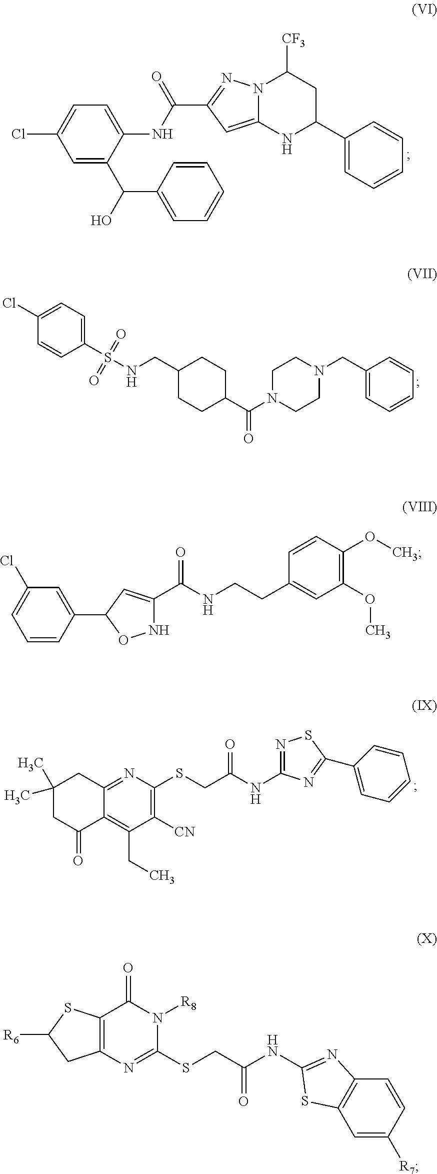

wherein:
   R₆ is hydrogen, alkyl_{(C≦8)}, or substituted alkyl_{(C≦8)},
   R₇ is hydrogen, alkyl_{(C≦8)} or substituted alkyl_{(C≦8)}; and
   R₈ is alkyl_{(C≦8)}, substituted alkyl_{(C≦8)}, aryl_{(C≦8)}, or substituted aryl_{(C≦8)};

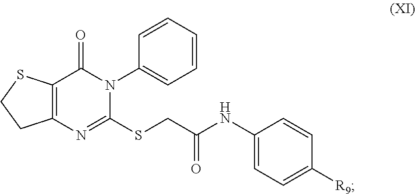

wherein:
   R₉ is acyl_{(C≦8)}, substituted acyl_{(C≦8)}, alkyl_{(C≦8)}, substituted alkyl_{(C≦8)} or
      heterocycloalkyl(C≦8) or substituted heterocycloalkyl(C≦8); or

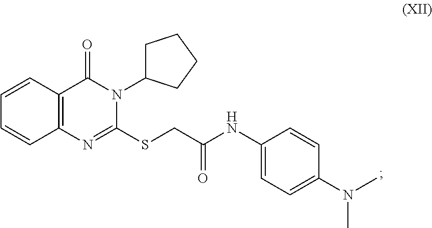 [SMILES: CN(C)c1ccc(NC(=O)CSc2nc3ccccc3c(=O)n2C2CCCC2)cc1]

or a pharmaceutically acceptable salt or tautomer thereof

The compound may be further defined as:

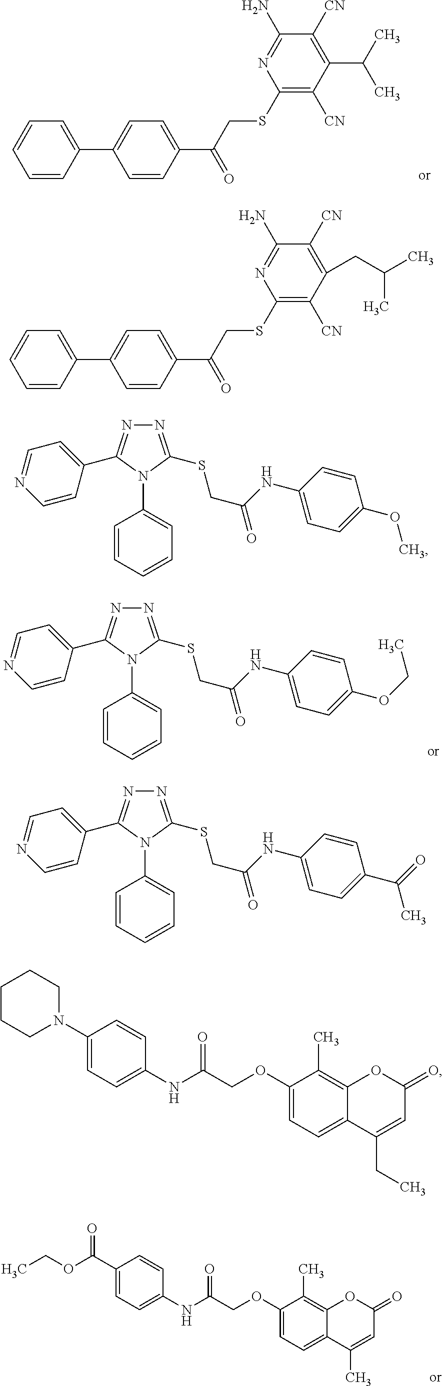

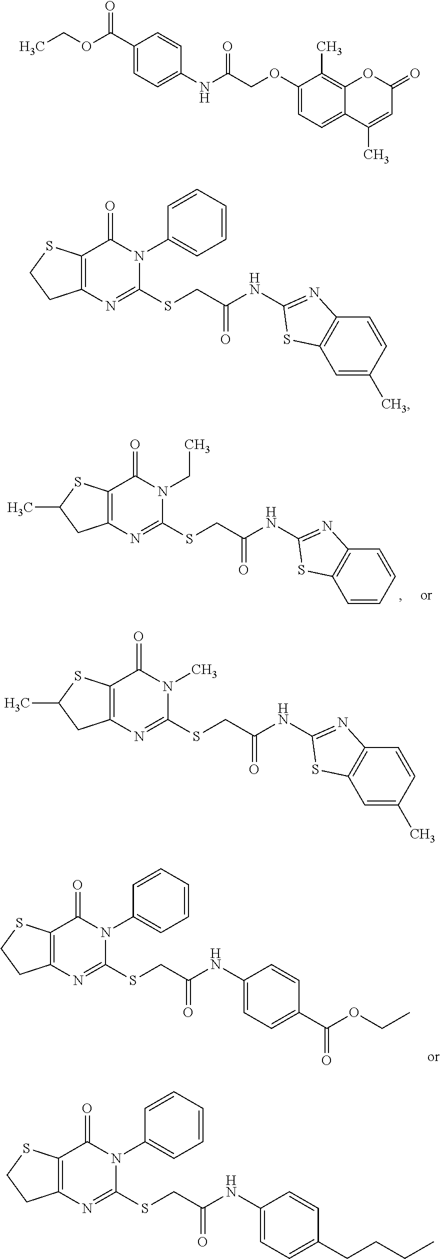

or a pharmaceutically acceptable salt or tautomer thereof. In some embodiments, the cell is in vitro. In other embodiments, the cell is in vivo. In some embodiments, the method of inhibiting Wnt protein signalling is further defined as a method of inhibiting Wnt response or Wnt production. In some embodiments, the method further comprises one of the specific compounds described above. The method may further comprise administering to said cell an inhibitor of a Tankyrase enzymes and/or a inhihitor of GSK-3β.

Methods of treatment are also contemplated by the present invention. Such methods may employ any compounds of the compounds described herein. For example, such methods may employ compounds of formulas described above and below. For example, the present invention contemplates a method of treating cancer in a patient comprising administering to the patient an effective amount of a compound of formula (II)-(XII), or any of the particular compounds set forth herein. For example, this includes any of the compounds disclosed in section III below, entitled “Wnt Protein Signalling Inhibitors.”

The cancer may be colorectal, breast, liver, lung, leukemia, pancreatic, renal, or prostate cancer. Methods of treating cancer may also further comprise administration of a chemotherapeutic, radiation therapy, immunotherapy, hormone therapy, toxin therapy, or gene therapy: such additional methodologies are well-known in the art. Methods of administration may include intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, subcutaneously, subconjunctival, intravesicularlly, mucosally, intrapericardially, intraumbilically, intraocularally, orally, locally, via inhalation, via injection, via infusion, via continuous infusion, via localized perfusion bathing target cells directly, via a catheter, via a lavage, in cremes, in lipid compositions, or any combination thereof. Dosages may include, e.g., about 1 μg/kg to about 100 mg/kg, or any range derivable therein.

In any method described herein, the compounds disclosed herein may be combined with a pharmaceutically acceptable carrier, diluent, and/or excipient in a pharmaceutical composition.

As noted above, pharmaceutical compositions are contemplated by the present invention. In certain embodiments, a pharmaceutical composition comprising a pharmaceutically acceptable carrier, diluent, and/or excipient and any one or more of the following compounds is contemplated:

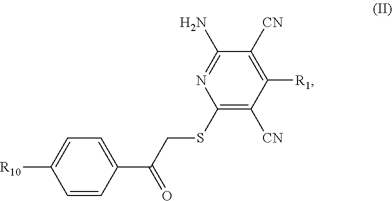

wherein:
   R₁ is alkyl_{(C≦8)} or substituted alkyl_{(C≦8)}; and
R₁₀ is aryl_{(C≦8)}, substituted aryl_{(C≦8)}, heterocycloalkyl_{(C≦8)} or substituted heterocycloalkyl_{(C≦8)};

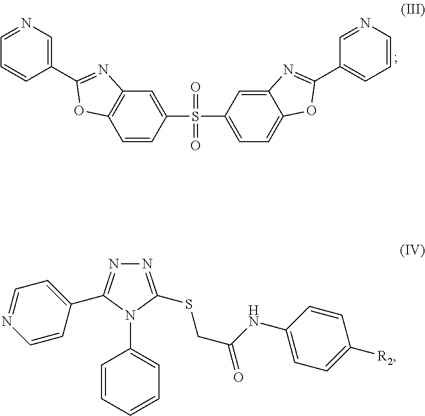

wherein R₂ is alkoxy_{(C≦8)}, substituted alkoxy_{(C≦8)}, acyl_{(C≦8)}, substituted acyl_{(C≦8)}; heterocycloalkyl_{(C≦8)} or substituted heterocycloalkyl_{(C≦8)};

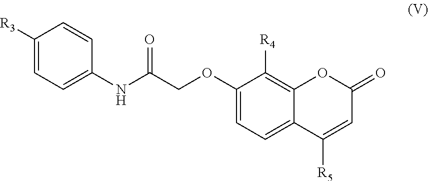

wherein:
   R₃ is acyl_{(C≦8)}, substituted acyl_{(C≦8)}, heterocycloalkylz_{(C≦8)} or substituted heterocycloalkyl_{(C≦8)};
   R₄ is hydrogen, alkyl_{(C≦8)} or substituted alkyl_{(C≦8)}; and
   R₅ alkyl_{(C≦8)} or substituted alkyl_{(C≦8)};

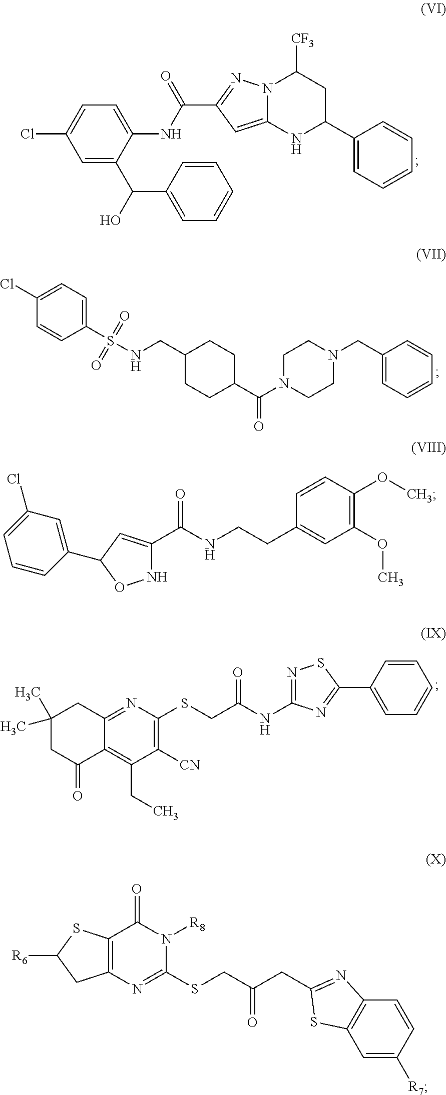

wherein:
   R₆ is hydrogen, alkyl_{(C≦8)}, or substituted alkyl_{(C≦8)};
   R₇ is hydrogen, alkyl_{(C≦8)} or substituted alkyl_{(C≦8)}; and
   R₈ is alkyl_{(C≦8)}, substituted alkyl_{(C≦8)}, aryl_{(C≦8)}, or substituted aryl_{(C≦8)};

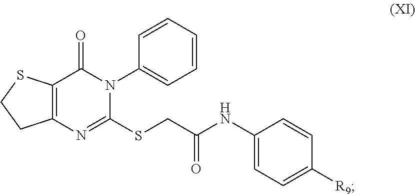

wherein:
   R₉ is acyl_{(C≦8)}, substituted acyl_{(C≦8)}, alkyl_{(C≦8)} substituted alkyl_{(C≦8)}, heterocycloalkyl_{(C≦8)} or substituted heterocycloalkyl_{(C≦8)}; or

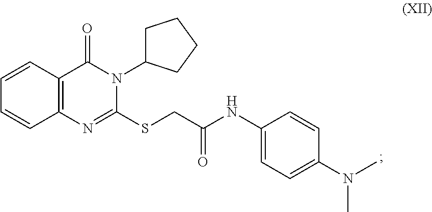 [SMILES: CN(C)c1ccc(NC(=O)CSc2nc3ccccc3c(=O)n2C2CCCC2)cc1]

or a pharmaceutically acceptable salt or tautomer thereof; or

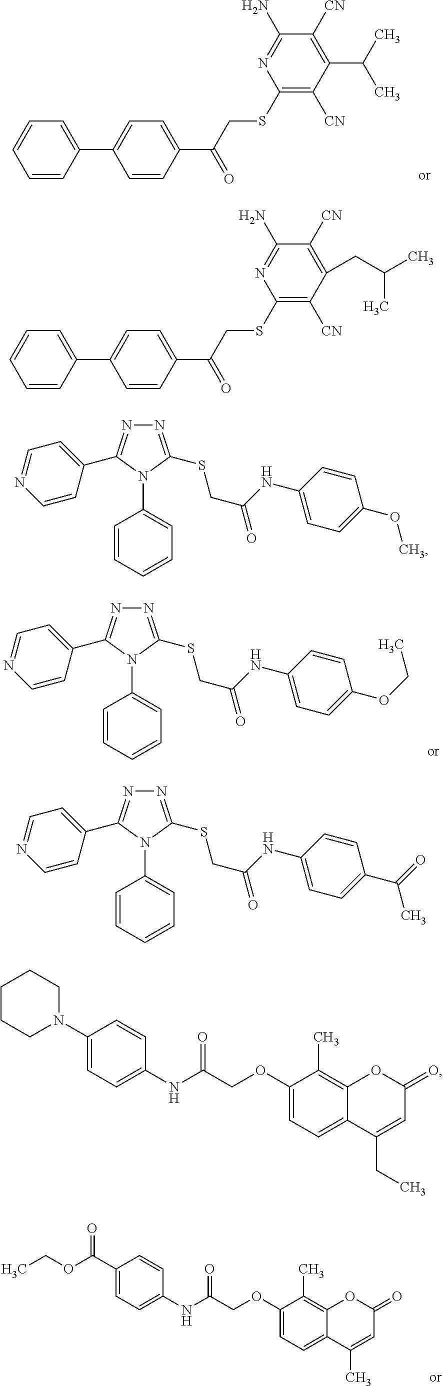

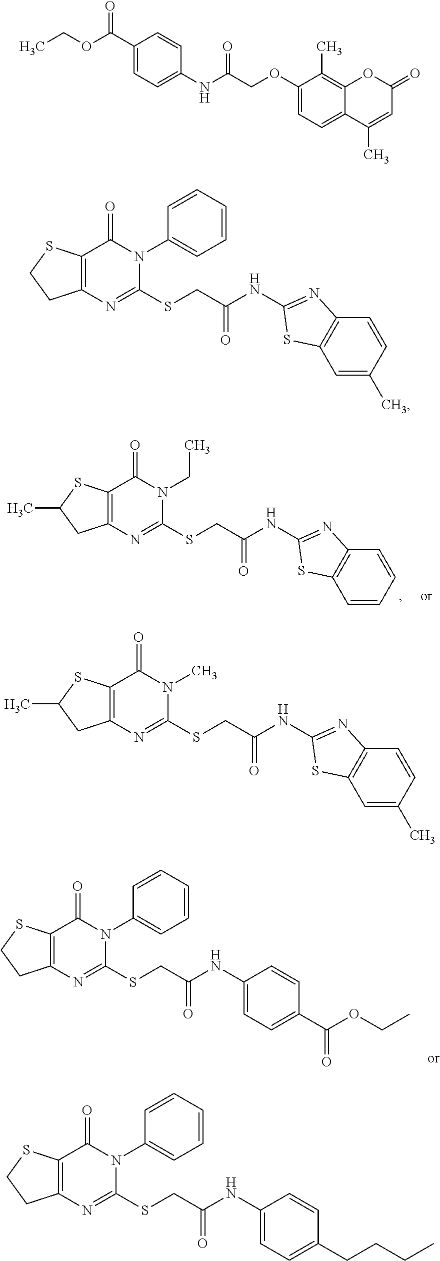

or a pharmaceutically acceptable salt or tautomer thereof

Another general aspect of the present invention contemplates a method of treating or preventing osteopetrosis in a patient comprising administering to the patient an effective amount of a compound disclosed herein. Such methods may further comprise administration of a second osteopetrosis-treating agent or a second osteopetrosis-preventing agent. Administration of the compound of interest may take place via a route selected from the group consisting of intravenously, intradermally, intraarterially, intraperitoneally, intralesionally, intracranially, intraarticularly, intranasally, topically, intramuscularly, subcutaneously, intraumbilically, orally, locally, via inhalation, via injection, via infusion, via continuous infusion, via localized perfusion bathing target cells directly, via a catheter, in cremes, in lipid compositions, or any combination thereof. Dosage amounts may range between, for example, about 1 μg/kg to about 100 mg/kg, or any range derivable therein. Treating may comprises slowing the onset or progression of osteoporosis.

Also contemplated by the present invention are methods of treating a degenerative disease in a patient comprising administering to the patient an effective amount of a compound disclosed herein. The degenerative disease may be, for example, type II diabetes or age-related impairment of tissue repair. Methods may further comprise administration of a second agent to treat the degenerative disease. Methods of administration may be selected from the group consisting of intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intramuscularly, subcutaneously, subconjunctival, intravesicularlly, mucosally, intrapericardially, intraumbilically, intraocularally, orally, locally, via inhalation, via injection, via infusion, via continuous infusion, via localized perfusion bathing target cells directly, via a catheter, via a lavage, in cremes, in lipid compositions, or any combination thereof. Dosage amounts may range between, for example, about 1 μg/kg to about 100 mg/kg, or any range derivable therein.

Also disclosed herein are methods of treating type II diabetes in a patient comprising administering to the patient an effective amount of a compound disclosed herein. Such methods may further comprise administration of a second agent to treat diabetes. Methods of administration may be selected from the group consisting of intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intramuscularly, subcutaneously, subconjunctival, intravesicularlly, mucosally, intrapericardially, intraumbilically, intraocularally, orally, locally, via inhalation, via injection, via infusion, via continuous infusion, via localized perfusion bathing target cells directly, via a catheter, via a lavage, in cremes, in lipid compositions, or any combination thereof. Dosage amounts may range between, for example, about 1 μg/kg to about 100 mg/kg, or any range derivable therein.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-D. The IWP compounds directly attack Porcn. (FIG. 1A) Synthesis of a fluorescently-labeled Porcn inhibitor. The IWP2 molecule was modified with a linker and Cy3 adduct to generate the IWP-Cy3 fluorescently labeled probe. (FIG. 1B) IWPCy3 specifically binds to Porcn. Cos-1 cells overexpressing Porcn or other members of the MBOAT family with recognized protein substrates (HHAT and GOAT) were treated with IWP-Cy3 and then gated for Cy3 staining Number of Cy3-positive cells in each experiment is normalized to the Porcn overexpression sample (=100%). (FIG. 1C) A Porcn mutant with an altered putative active site residue (Porcn H335D) does not engage IWP-Cy3. Wild-type Porcn or Porcn H335D sequence fused to Gaussia luciferase DNA (to stabilize the Porcn H335D protein; see FIG. S2) was transfected into Cos1 cells and IWP-Cy3-association determined as before. Competition with unlabeled IWP2 serves as a specificity control for IWP-Cy3 binding. (FIG. 1D) Expression of Porcn H335D-GL protein is not significantly affected by IWP2. Western blot analysis of wt and H335D Porcn in Cos1 cells in the presence or absence of IWP2.

FIGS. 2A-B. The IWP compounds specifically inhibit Porcn acyltransferase activity. (FIG. 2A) IWP2 inhibits Wnt fatty acylation. Cells transfected with an expression construct expressing either a fusion molecule consisting of Wnt3A and the Fc region of human IgG (Wnt3A-Fc) or IgG-Fc (Fc) alone are treated with C16 ω-alkynyl fatty acid (alkynyl-PA). Purified alkynyl-PA-labeled fusion protein bound to Protein A sepharose is treated with biotin-azide reagent which enables protein detection using streptavidin-HRP. RNAi-mediated knock-down or overexpression of Porcn respectively results in loss or increase in Wnt3A-Fc protein labeling with alkynyl-PA. IWP2 is able to block the labeling of Wnt3A. (FIG. 2B) IWP2 does not inhibit Hh fatty acylation. The same click chemistry strategy is used to monitor fatty acylation of Hh protein. IWP2 does not block Hh labeling with alkynyl-PA.

FIGS. 3A-D. The IWP compounds target both β-catenin-dependent and -independent Wnt pathway responses. (FIG. 3A) IWP2 inhibits the secretion of Wnt1 protein in embryonic kidneys. Urogenital systems from E11.5 mice expressing Wnt1-GFP were removed, bisected, and treated with IWP2 in vitro for 24 hours. A cross section of ureteric bud (top panels) or the total organ (bottom) was analyzed for Wnt1-GFP expression in order to determine its localization and to visualize branching tissues, respectively. β-catenin staining is used here to reveal cellular boundaries (β-catenin bound to the β-cadherin cell-cell adhesion receptor). (FIG. 3B) IWP2 inhibits the secretion of Wnt proteins regardless of their ability to induce transcriptional responses in HEK293 cells. The secretion of several Wnt-GL fusion proteins introduced by DNA transfection into HEK293 cells was tested for sensitivity to IWP2 (top). In parallel, the ability of the same Wnt molecules lacking GL to activate Wnt/β-catenin pathway response as measured using the STF reporter was determined (bottom). Data are mean+SEM from three measurements. (FIG. 3C) Evidence that IWP2 inhibits the production of a non-canonical Wnt (Wnt5A). Antagonism of Wnt/β-catenin signaling by expression of Wnt5A in the presence or absence of IWP compound was determined in HEK293 cells transfected with the STF reporter and treated with Wnt3A-containing conditioned medium. STF activity was normalized to the activity of a co-transfected control reporter. Data are mean+SEM from three measurements. P values for change from control response are indicated. (FIG. 3D) IWP inhibits Wnt-dependent activation of Jnk. Mouse L fibroblasts transfected with Wnt7B DNA induce IWP-sensitive phosphorylation of Jnk, a target of multiple β-catenin-independent Wnt pathways.

FIGS. 4A-E. Diverse chemical scaffolds support Porcn inhibition by targeting the putative active site. (FIG. 4A) Dv12 phosphorylation status in HeLa cells reflects Porcn activity. IWP2 inhibits Dv12 phosphorylation in HeLa cells indicating cell-autonomous Wnt-mediated signaling in these cells as previously described. (FIG. 4B) Identification of additional Porcn inhibitors. The IWP compound collection of Wnt/β-catenin pathway inhibitors was tested for their ability to inhibit Dv12phosphorylation in HeLa cells. The ratio of phosphorylated to unphosphorylated Dv1 protein in cells treated with each IWP compound was determined by densitometric analysis of Western blot results as shown in FIG. 4A. Compounds inhibiting 90% or more of Dv1 phosphorylation are labeled. (FIG. 4C) Shared chemical scaffolds yielding the most active IWP molecules. Compounds are clustered based on their similarity to IWP2 or shared chemical structures. IC₅₀ against Wnt/β-catenin pathway response as measured by STF is provided for at least one representative compound from each class. (FIG. 4D) Novel IWP compounds disrupt Wnt protein acylation. Wnt3A-Fc protein from cells treated with alkynyl-PA in the presence of indicated IWP compound or DMSO was subjected to an alkyne cycloaddition reaction to label fatty acylated Wnt3A with biotin. Biotinylated protein separated on SDS-PAGE was visualized with streptavidin HRP. (FIG. 4E) Novel Porcn inhibitors likely bind directly to Porcn. The ability of indicated IWP compounds to compete for IWP-Cy3 binding to Porcn was determined as before.

FIGS. 5A-F. Concerted deployment of IWP and IWR compounds distinguishes 3-catenin-dependent and -independent responses in vivo. (FIG. 5A) Identification of an IWP compound with in vivo activity in zebrafish. IWP12 inhibits the expression of an EGFP fluorescent protein reporter driven by synthetic TCF-binding elements in a transgenic line [Tg(7xTCF-X1a.Siam:GFP)ia4]. An approximately 10 fold excess of IWP12 is equivalent in activity to IWR1 compound. Fluorescence intensity was quantified (below) in an area that covers most of the posterior region (box). Data are mean+SEM from three animals. (FIG. 5B) IWR and IWP compounds inhibit Wnt signaling in zebrafish primary embryonic fibroblasts. Embryonic fibroblasts isolated from 6 hpf Tg(7xTCF-X1a.Siam:GFP)ia4 embryos were cultured in the presence or absence of indicated compound. GFP expression was visualized 20 hrs later. (FIG. 5C) IWP compounds inhibit tailfin regeneration, a Wnt-dependent process. Tailfins of zebrafish larvae at 3 days post fertilization were resected and the larvae subsequently reared in medium containing DMSO, IWR1 (10 μM), or IWP12 (50 μM) for an additional 4 days. (FIG. 5D) IWP12 inhibits embryonic convergent extension by targeting β-catenin-independent Wnt signaling. Zebrafish embryos were treated with Gsk3β inhibitor (a Wnt/β-catenin pathway activator), IWP12 compound, or both starting 4 hpf followed by whole mount in situ analysis at 24 hpf with probes and the respective developmental structures they label indicated: hgg1 (cts11b) (prechordal plate (pcp)), nt1 (prospective notochord (n) and germ ring blastopore margin), and d1x3b (anterior edge of the neural plate (np)). Changes in the distance between the neural plates and prechordal plates, as well as the notochord were quantified based upon the severity of the phenotype as represented. Number of animals examined under each condition is indicated above each plot. (FIG. 5E) Inactivation of Gsk3β rescues Wnt/β-catenin pathway activity in animals treated with IWP12. (FIG. 5F) Engrailed expression in the midbrain/hindbrain boundary (MHB) is suppressed by chemical inhibition of Porcn. Zebrafish embryos (4 hpf) treated with IWP 12 for 20 hours were subjected to in situ analysis with a probe for eng1a. Number of animals examined in each condition is indicated within each plot.

FIG. 6. Synthetic scheme for IWP-Cy3.

FIGS. 7A-B. The activity of Porcn-Gaussia luciferase (GL) fusion proteins is a faithful reporter of Porcn function. (FIG. 7A) Porcn-GL activity is similarly sensitive to mutations that influence activity of a Myc-epitope tagged Porcn protein. The indicated Porcn-GL and -Myc proteins were tested for their ability to counter the effects of IWP2 on Wnt/β-catenin pathway response as measured using the SuperTopFlash (STF) reporter in HEK293 cells. (FIG. 7B). Both Porcn-myc and Porcn-GL proteins exhibit a reticular, intracellular expression pattern consistent with previous assignment of Porcn localization to the ER.

FIG. 8. Identification of novel IWP compounds that inhibit Dv1 protein phosphorylation. HeLa cells treated with various IWP compounds identified by Chen et al. (2009) were analyzed by Western blotting for Dv12 protein phosphorylation status to identify novel inhibitors of Wnt activity.

FIG. 9. Identification of potent inhibitors of Wnt activity. The potency of representative IWP compounds from different scaffold classes identified from using the Dv1 protein phosphorylation assay was determined by further testing of Wnt/β-catenin pathway inhibitory activity in L-Wnt-STF cells (Wnt3A-expressing L fibroblasts harboring the STF and a control reporter).

FIGS. 10. Dose-dependent effects of IWR1 and IWP12 on Wnt/β-catenin pathway response in zebrafish embryos. The effects of Wnt pathway inhibitors added to aquarium water on the activity of the Wnt/β-catenin pathway reporter in the Tg(7xTCF-X1a.Siam:GFP)ia4 fish were determined Fluorescence intensity of EGFP was quantified (below each image) in an area that covers most of the posterior region as before. Data are mean+SEM from three animals.

### DESCRIPTION OF ILLUSTRATIVE EMBODIMENTS

Chemically-based strategies are ideally suited for studying the molecular basis of complex biological phenomena given the potential of small molecules to overcome some of these limitations. Previously, the inventors described two classes of small molecules that disengage Wnt-mediated responses (Chen et al., 2009). The Inhibitors of Wnt Response (IWR) compounds target the Tankyrase (Tnks) enzymes that regulate Axin protein turnover, scaffolding molecules in the β-catenin destruction complex (Chen et al., 2009; Huang and He, 2008). In the absence of Tnks activity, Axin proteins accumulate and accelerate the rate of β-catenin destruction. On the other hand, the Inhibitor of Wnt Production (IWP) compounds disrupt Wnt signaling by preventing Porcndependent lipidation of Wnt proteins. Porcn is the founding member of the membrane bound Oacyltransferase (MBOAT) family that consists of 16 family members (Yang et al., 2008). Likely due to their limited bioavailability, the IWP compounds, unlike the IWR compounds, did not exhibit in vivo activity (Chen et al., 2009). Instead, IWP compounds have been extensively used in a variety of in vitro settings for tissue engineering and stem cell biology (Ren et al., 2011; Sato et al., 2011; ten Berge et al., 2011).

In order to expand the utility of Porcn inhibitors to include in vivo studies, the inventors have identified additional Porcn compounds from screening a small collection of Wnt pathway inhibitors with no previously assigned target. They demonstrate that all of these compounds directly engage Porcn at its putative active site thus revealing Porcn to be a highly druggable enzyme. Using one of these novel Porcn inhibitors (IWP12) in concert with other Wnt pathway modulators, the now provide evidence for a role of Wnt protein lipidation in promoting diverse Wnt-mediated responses in development and tissue regeneration, and establish a chemical toolkit for interrogating Wnt signaling mechanisms in these contexts. Small molecules that target Wnt-dependent signal transduction pathways reveal chemically-sensitive regulatory mechanisms within these signal transduction pathway that may be exploited by pharmacological means for medical use, such as regenerative and anti-cancer therapy.

### I. THE WNT SIGNAL TRANSDUCTION PATHWAYS

The Wnt gene family encodes secreted ligand proteins that serve key roles in differentiation and development. This family comprises at least 15 vertebrate and invertebrate genes including the Drosophila segment polarity gene wingless and one of its vertebrate homologues, integrated from which the Wnt name derives. As noted above, the Wnt proteins appear to facilitate a number of developmental and homeostatic processes.

The Wnt signalling pathways comprises a number of proteins involved in the transduction of cellular responses to secreted Wnt/wingless signalling proteins. Wnt proteins that control “non-canonical” pathways, such as the Wnt/calcium and planar cell polarity pathways, induce cellular responses that are not dependent upon β-catenin. In the Wnt/β-catenin pathway, the Frizzled receptor then activates Disheveled protein, which blocks the inhibiting action of Zeste-white-3 kinase (or GSK3β in vertebrates, Glycogen Synthase Kinase-3β) upon the Armadillo protein (a β-catenin protein). The β-catenin protein transduces the Wnt signal from the cytoplasm to the nucleus. In the absence of Wnt signalling, β-catenin is constitutively degraded by the proteasome and can be found in a multimeric complex with conductin (or axin), APC (Adenomatous Polyposis Coli) and GSK3β. APC mediates the binding of β-catenin to conductin and serves to activate the conductin protein. Conductin acts as a scaffold to assemble the components of the degradation pathway of β-catenin. GSK3β, a serine/threonine kinase, phosphorylates β-catenin, thus stimulating its degradation by the proteasome.

Upon Wnt signalling, GSK3β kinase is inactivated, leading to stabilization of the -catenin protein. β-Catenin is then released from the multimeric complex and translocates into the nucleus. Once in the nucleus, β-catenin interacts with the LEF/TCF (Lymphoid Enhancer Factor/T-Cell Factor) family of HMG (High Mobility Group) box transcription factors. The LEF/TCF factors are stimulated through interaction with β-catenin to become potent transactivators of a number of genes including c-myc and cyclin Dl.

### II. THERAPEUTIC IMPLICATIONS OF WNT-CONTROLLED SIGNAL TRANSDUCTION PATHWAYS

As noted above, evidence suggests that targeting the Wnt-mediated signal transduction pathways would be therapeutically useful in a broad range of diseases (Barker and Clevers, 2006) (Veeman et al, 2003). Aged mice or mice that exhibit premature stem cell senescence that are treated with extracellular protein inhibitors of Wnt pathways exhibit improved regenerative capacity in various tissues (Brack et al., 2007; Liu et al., 2007). Mutations leading to constitutive activation of the Wnt pathway are critical events in a variety of human cancers including colon cancer, melanoma, hepatocellular carcinoma and others. The end result of constitutive activation of the Wnt/β-catenin pathway is a dramatic increase in the level of β-catenin protein in the cytoplasm. Inappropriate stabilization of -catenin, leading to increased levels of the protein, can be caused by mutations in a variety of proteins in the Wnt signalling pathway. Blockade of the Wnt/β-catenin pathway in a variety of cancers using either genetic or chemical approaches been shown to abrogate aberrant cell growth (Barker and Clevers, 2006). Furthermore, inhibition of this pathway may directly influence the cells that sustain cancer cell growth and enable metastasis, and that are thought to be resistant to traditional chemotherapeutic agents (Ailles and Weissman, 2007).

The pervasive influence of the Wnt proteins in tissue homeostasis and tumorigenesis suggests areas such as regenerative medicine and anti-cancer therapy may benefit from therapies that target this pathway. Achieving transient repression of pathological Wnt response without incurring permanent damage to normal stem cell function is a key anticancer therapeutic goal. The inventors tested for the ability of zebrafish to resume regenerative processes following a chemically induced blockade of fin regrowth. Fish with resected caudal fins that were bred in water containing IWR-1 for 7 d were able to regenerate tissue to nearly normal levels after chemical removal, which suggests that transient inhibition of Wnt/β-catenin response does not permanently alter the ability of stem cells to self-renew.

Aberrant Wnt-mediated pathway responses, sustained by genetic changes that result either in altered Wnt ligand activity or in altered functioning of pathway regulators, have been associated with a broad range of cancers. See Clevers, 2006 and Polakis, 2007, both of which are incorporated herein by reference. Notably, more than 90% of colorectal cancer (CRC) tumors harbor a loss-of-function mutation in APC, a suppressor of the Wnt/β-catenin pathway. See Sjoblom et al., 2006, which is incorporated herein by reference. The ability of IWR compounds to stabilize Axin proteins and induce β-catenin destruction even in the absence of normal APC protein function suggests that they may block aberrant cell growth supported by hyperactivation of Wnt/β-catenin responses.

Indeed, IWR compounds are able to inhibit aberrant Wnt/β-catenin activity as a consequence of Apc loss in both mouse L cells (using Apc small interfering RNAs) and DLD-1 colorectal cancer cells (that harbor a loss-of-function mutation in APC). The ability of IWR-3 to mimic the cell growth effects of β-catenin siRNAs in several cancer cell lines that exhibit differences in growth dependency on Wnt/β-catenin pathway activity was also tested. Notably, IWR-3 mimicked the effects of b-catenin siRNAs on the growth of cells derived from cancers of the colon (DLD-1) and prostate (DU145) but not lung (H460), which suggests that IWR-3 successfully targeted the Wnt/β-catenin pathway in these cells. Indeed, overexpression of b-catenin can overcome the effects of IWR-3 on DLD-1 cell growth.

Aberrant transcriptional induction of Wnt/β-catenin target genes is typically observed in CRC cells that harbor loss-of-function mutations in the APC tumor suppressor. Consistent with the ability of IWR compounds to inhibit cancerous Wnt/β-catenin pathway responses, a decrease in the expression of Axin2 in DLD-1 cells after exposure to IWR-1 for 2 h was observed. Thus, Axin protein stability can be chemically controlled in order to suppress cancerous Wnt/b-catenin activity, as demonstrated by IWR compounds.

### III. WNT PROTEIN SIGNALLING INHIBITORS

Accordingly, the present invention provides small molecules that inhibit the Wnt protein signalling pathway. These compounds are represented by the formulas:

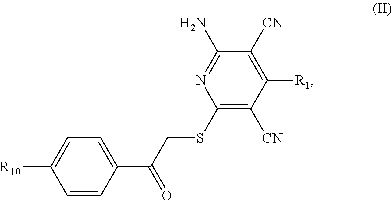

wherein:
   R₁ is alkyl_{(C≦8)} or substituted alkyl_{(C≦8)}; and
   R₁₀ is aryl_{(C≦8)}, substituted aryl_{(C≦8)}, heterocycloalkyl_{(C≦8)} or substituted heterocycloalkyl_{(C≦8)};

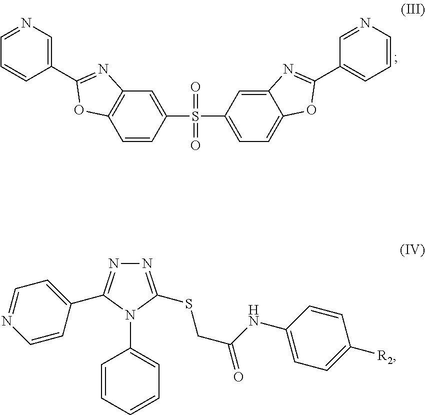

wherein R₂ is alkoxy_{(C≦8)}, substituted alkoxy_{(C≦8)}, acyl_{(C≦8)}, substituted acyl_{(C≦8)}, heterocycloalkyl_{(C≦8)} or substituted heterocycloalkyl_{(C≦8)};

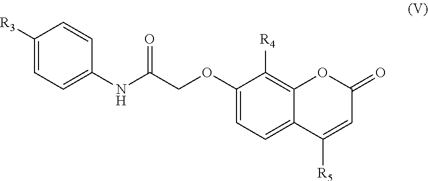

wherein:
R₃ is acyl_{(C≦8)}, substituted acyl_{(C≦8)}, heterocycloalkyl_{(C≦8)} or substituted heterocycloalkyl_{(C≦8)};
R₄ is hydrogen, alkyl_{(C≦8)} or substituted alkyl_{(C≦8)}; and
R₅ alkyl_{(C≦8)} or substituted alkyl_{(C≦8)};

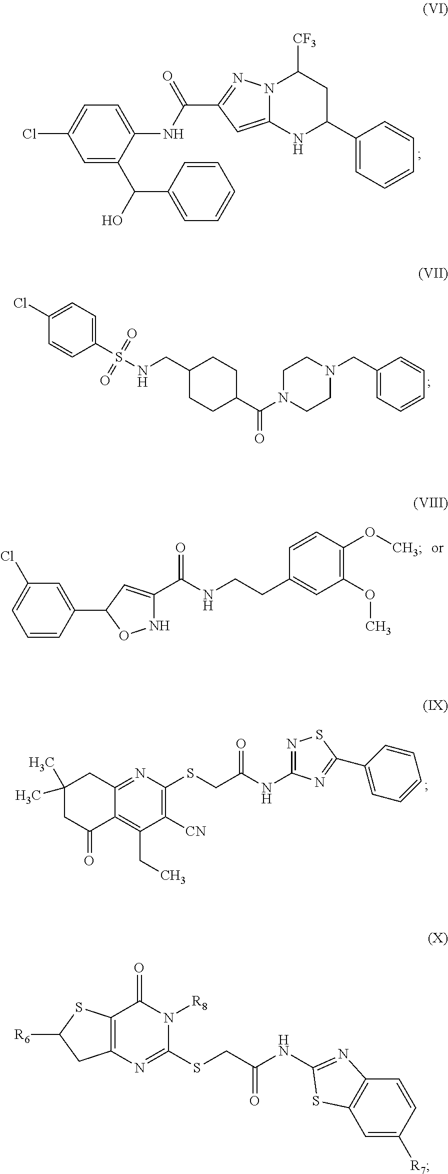

wherein:
   R₆ is hydrogen, alkyl_{(C≦8)}, or substituted alkyl_{(C≦8)};
   R₇ is hydrogen, alkyl_{(C≦8)} or substituted alkyl_{(C≦8)}; and
   R₈ is alkyl_{(C≦8)}, substituted alkyl_{(C≦8)}, aryl_{(C≦8)}, or substituted aryl_{(C≦8)};

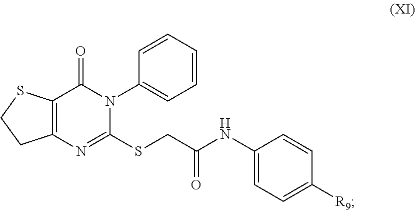

wherein:
   R₉ is acyl_{(C≦8)}, substituted acyl_{(C≦8)}, alkyl_{(C≦8)}, substituted alkyl_{(C≦8)}, heterocycloalkyl_{(C≦8)} or substituted heterocycloalkyl_{(C≦8)}; or

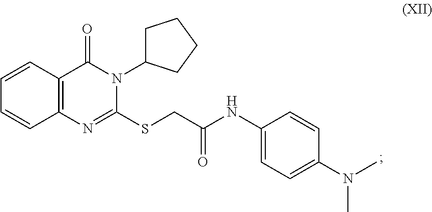 [SMILES: CN(C)c1ccc(NC(=O)CSc2nc3ccccc3c(=O)n2C2CCCC2)cc1]

or a pharmaceutically acceptable salt or tautomer thereof. Particular compounds include:

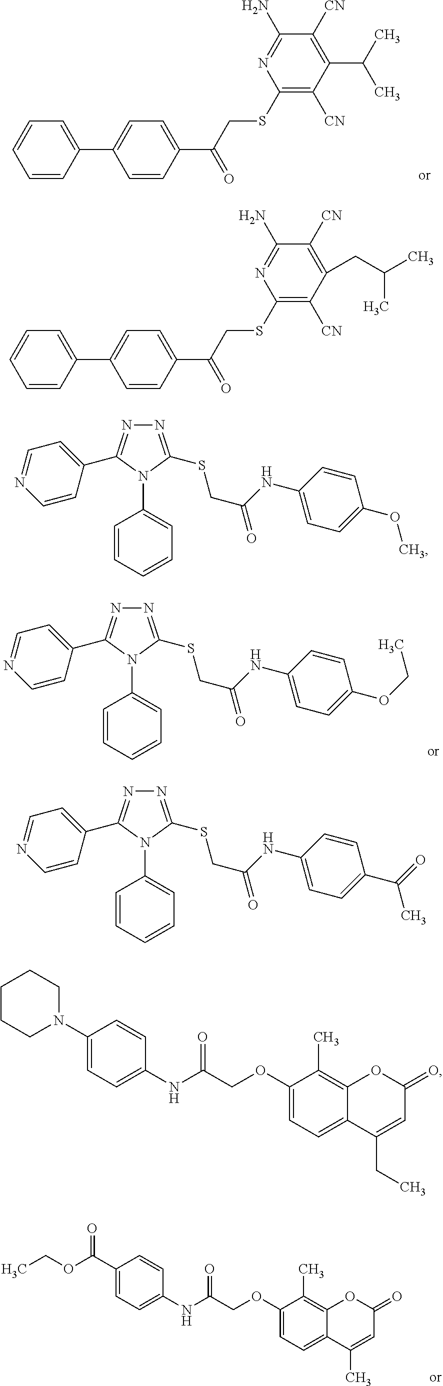

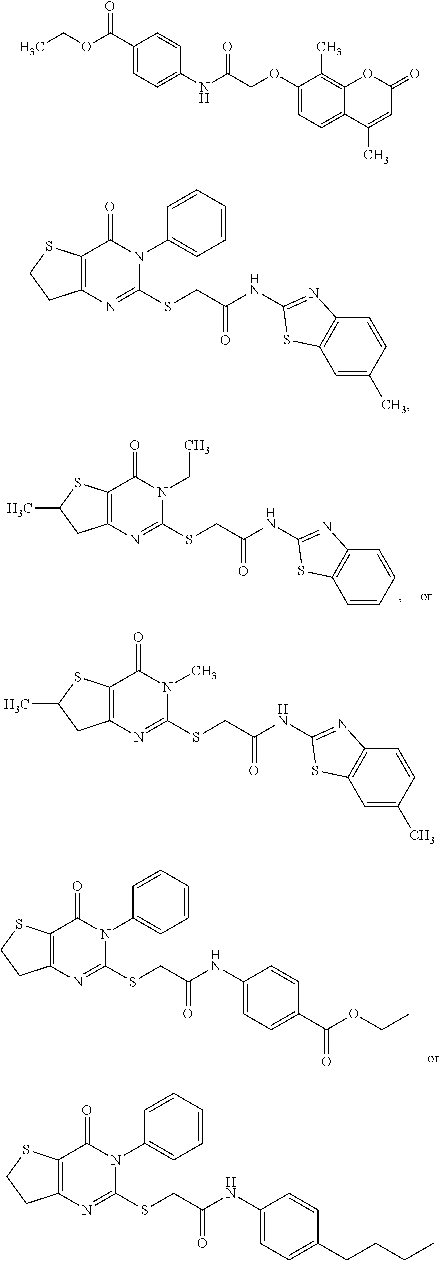

or a pharmaceutically acceptable salt or tautomer thereof

All of these methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

Compounds of the invention may contain one or more asymmetrically-substituted carbon or nitrogen atoms, and may be isolated in optically active or racemic form. Thus, all chiral, diastereomeric, racemic form, epimeric form, and all geometric isomeric forms of a structure are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present invention can have the S or the R configuration.

Compounds of the invention may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C. Similarly, it is contemplated that one or more carbon atom(s) of a compound of the present invention may be replaced by a silicon atom(s). Furthermore, it is contemplated that one or more oxygen atom(s) of a compound of the present invention may be replaced by a sulfur or selenium atom(s).

Compounds of the present invention may also exist in prodrug form. Since prodrugs are known to enhance numerous desirable qualities of pharmaceuticals (e.g., solubility, bioavailability, manufacturing, etc.), the compounds employed in some methods of the invention may, if desired, be delivered in prodrug form. Thus, the invention contemplates prodrugs of compounds of the present invention as well as methods of delivering prodrugs. Prodrugs of the compounds employed in the invention may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a subject, cleaves to form a hydroxy, amino, or carboxylic acid, respectively.

It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

### IV. DEFINITIONS

As used herein, “Wnt protein signalling pathway” refers to the pathways by which binding of the Wnt protein to extracellular receptors is either translated into the nucleus and results in transcriptional activation of a variety of genes, or otherwise results in biochemical changes that influence cell behavior. The Wnt protein signalling pathways involve a variety of proteins including Frizzled, Disheveled, Axin, APC, GSK3β, β-catenin, LEF/TCF transcription factors, etc. Cells from many different species express homologs of the proteins involved in Wnt protein signalling pathways and accordingly have functionally equivalent Wnt protein signalling pathways.

As used herein, a “Wnt protein signalling inhibitor” is an organopharmaceutical (that is, a small organic molecule) that inhibits Wnt protein signalling activity. Wnt protein signalling inhibitors typically have a molecular weight of about 1000 g/mol or less.

As used herein, “a method of inhibiting Wnt response” refers to methods of inhibiting known biochemical events associated with production of functional Wnt proteins or with cellular responses to Wnt proteins. As discussed herein, small organic molecules may inhibit Wnt response in accordance with this definition.

When used in the context of a chemical group, “hydrogen” means —H; “hydroxy” means —OH; “oxo” means ═O; “halo” means independently —F, —Cl, —Br or —I; “amino” means —NH₂; “hydroxyamino” means —NHOH; “nitro” means —NO₂; imino means ═NH; “cyano” means —CN; “isocyanate” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “sulfonyl” means —S(O)₂—; and “sulfinyl” means —S(O)—.

In the context of chemical formulas, the symbol “—” means a single bond, “═” means a double bond, and “≡” means triple bond. The symbol “----” represents an optional bond, which if present is either single or double. The symbol “ [symbol] ” represents a single bond or a double bond. Thus, for example, the structure

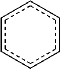

includes the structures

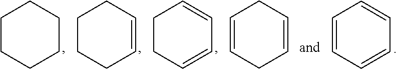

As will be understood by a person of skill in the art, no one such ring atom forms part of more than one double bond. The symbol “ [symbol] ”, when drawn perpendicularly across a bond indicates a point of attachment of the group. It is noted that the point of attachment is typically only identified in this manner for larger groups in order to assist the reader in rapidly and unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the conformation (e.g., either R or S) or the geometry is undefined (e.g., either E or Z).

Any undefined valency on an atom of a structure shown in this application implicitly represents a hydrogen atom bonded to the atom. When a group “R” is depicted as a “floating group” on a ring system, for example, in the formula:

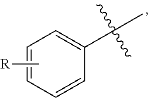

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

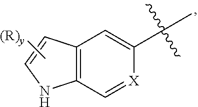

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the groups and classes below, the following parenthetical subscripts further define the group/class as follows: “(Cn)” defines the exact number (n) of carbon atoms in the group/class. “(C≦n)” defines the maximum number (n) of carbon atoms that can be in the group/class, with the minimum number as small as possible for the group in question, e.g., it is understood that the minimum number of carbon atoms in the group “alkenyl_{(C≦8)}” or the class “alkene_{(C≦8)}” is two. For example, “alkoxy_{(C≦10)}” designates those alkoxy groups having from 1 to 10 carbon atoms (e.g., 1, 2, 3, 4, 5, 6, 7, 8, 9, or 10, or any range derivable therein (e.g., 3 to 10 carbon atoms). (Cn-n′) defines both the minimum (n) and maximum number (n′) of carbon atoms in the group. Similarly, “alkyl_{(C2-10)}” designates those alkyl groups having from 2 to 10 carbon atoms (e.g., 2, 3, 4, 5, 6, 7, 8, 9, or 10, or any range derivable therein (e.g., 3 to 10 carbon atoms)).

The term “saturated” as used herein means the compound or group so modified has no carbon-carbon double and no carbon-carbon triple bonds, except as noted below. The term does not preclude carbon-heteroatom multiple bonds, for example a carbon oxygen double bond or a carbon nitrogen double bond. Moreover, it does not preclude a carbon-carbon double bond that may occur as part of keto-enol tautomerism or imine/enamine tautomerism.

The term “aliphatic” when used without the “substituted” modifier signifies that the compound/group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single bonds (alkanes/alkyl), or unsaturated, with one or more double bonds (alkenes/alkenyl) or with one or more triple bonds (alkynes/alkynyl). When the term “aliphatic” is used without the “substituted” modifier only carbon and hydrogen atoms are present. When the term is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —OC(O)CH₃, or —S(O)₂NH₂.

The term “alkyl” when used without the “substituted” modifier refers to a monovalent saturated aliphatic group with a carbon atom as the point of attachment, a linear or branched, cyclo, cyclic or acyclic structure, and no atoms other than carbon and hydrogen. Thus, as used herein cycloalkyl is a subset of alkyl. The groups —CH₃ (Me), —CH₂CH₃ (Et), —CH₂CH₂CH₃ (n-Pr), —CH(CH₃)₂ (iso-Pr), —CH(CH₂)₂ (cyclopropyl), —CH₂CH₂CH₂CH₃ (n-Bu), —CH(CH₃)CH₂CH₃ (sec-butyl), —CH₂CH(CH₃)₂ (iso-butyl), —C(CH₃)₃ (tert-butyl), —CH₂C(CH₃)₃ (neo-pentyl), cyclobutyl, cyclopentyl, cyclohexyl, and cyclohexylmethyl are non-limiting examples of alkyl groups. The term “alkanediyl” when used without the “substituted” modifier refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, —CH₂CH₂CH₂—, and

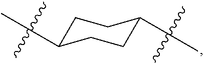 [SMILES: *C1CCC(*)CC1]

are non-limiting examples of alkanediyl groups. The term “alkylidene” when used without the “substituted” modifier refers to the divalent group ═CRR′ in which R and R′ are independently hydrogen, alkyl, or R and R′ are taken together to represent an alkanediyl having at least two carbon atoms. Non-limiting examples of alkylidene groups include: ═CH₂, ═CH(CH₂CH₃), and ═C(CH₃)₂. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —OC(O)CH₃, or —S(O)₂NH₂. The following groups are non-limiting examples of substituted alkyl groups: —CH₂OH, —CH₂C1, —CF₃′ —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂C1. The term “haloalkyl” is a subset of substituted alkyl, in which one or more hydrogen atoms has been substituted with a halo group and no other atoms aside from carbon, hydrogen and halogen are present. The group, —CH₂C1 is a non-limiting examples of a haloalkyl. An “alkane” refers to the compound H—R, wherein R is alkyl. The term “fluoroalkyl” is a subset of substituted alkyl, in which one or more hydrogen has been substituted with a fluoro group and no other atoms aside from carbon, hydrogen and fluorine are present. The groups, —CH₂F, —CF₃, and —CH₂CF₃ are non-limiting examples of fluoroalkyl groups. An “alkane” refers to the compound H—R, wherein R is alkyl.

The term “alkenyl” when used without the “substituted” modifier refers to an monovalent unsaturated aliphatic group with a carbon atom as the point of attachment, a linear or branched, cyclo, cyclic or acyclic structure, at least one nonaromatic carbon-carbon double bond, no carbon-carbon triple bonds, and no atoms other than carbon and hydrogen. Non-limiting examples of alkenyl groups include: —CH═CH₂ (vinyl), —CH═CHCH₃, —CH═CHCH₂CH₃, —CH₂CH═CH₂ (allyl), —CH₂CH═CHCH₃, and —CH═CH—C₆H₅. The term “alkenediyl” when used without the “substituted” modifier refers to a divalent unsaturated aliphatic group, with two carbon atoms as points of attachment, a linear or branched, cyclo, cyclic or acyclic structure, at least one nonaromatic carbon-carbon double bond, no carbon-carbon triple bonds, and no atoms other than carbon and hydrogen. The groups, —CH═CH—, —CH═C(CH₃)CH₂—, —CH═CHCH₂—, and

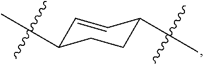 [SMILES: *C1C=CC(*)CC1]

are non-limiting examples of alkenediyl groups. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —OC(O)CH₃, or —S(O)₂NH₂. The groups, —CH═CHF, —CH═CHCl and —CH═CHBr, are non-limiting examples of substituted alkenyl groups. An “alkene” refers to the compound H—R, wherein R is alkenyl.

The term “alkynyl” when used without the “substituted” modifier refers to an monovalent unsaturated aliphatic group with a carbon atom as the point of attachment, a linear or branched, cyclo, cyclic or acyclic structure, at least one carbon-carbon triple bond, and no atoms other than carbon and hydrogen. As used herein, the term alkynyl does not preclude the presence of one or more non-aromatic carbon-carbon double bonds. The groups, —C≡CH, —C≡CCH₃, and —CH₂C≡CCH₃, are non-limiting examples of alkynyl groups. When alkynyl is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —OC(O)CH₃, or —S(O)₂NH₂. An “alkyne” refers to the compound H—R, wherein R is alkynyl.

The term “aryl” when used without the “substituted” modifier refers to a monovalent unsaturated aromatic group with an aromatic carbon atom as the point of attachment, said carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl group (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and the monovalent group derived from biphenyl. The term “arenediyl” when used without the “substituted” modifier refers to a divalent aromatic group, with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl group (carbon number limitation permitting) attached to the first aromatic ring or any additional aromatic ring present. If more than one ring is present, the rings may be fused or unfused. Non-limiting examples of arenediyl groups include:

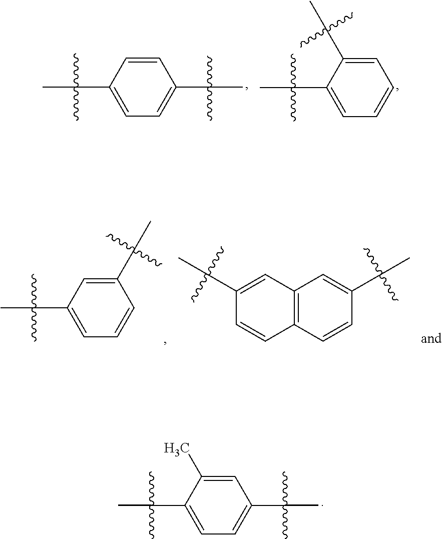

When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —OC(O)CH₃, or —S(O)₂NH₂. An “arene” refers to the compound H—R, wherein R is aryl.

The term “aralkyl” when used without the “substituted” modifier refers to the monovalent group -alkanediyl-aryl, in which the terms alkanediyl and aryl are each used in a manner consistent with the definitions provided above. Non-limiting examples of aralkyls are: phenylmethyl (benzyl, Bn) and 2-phenyl-ethyl. When the term is used with the “substituted” modifier one or more hydrogen atom from the alkanediyl and/or the aryl has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —OC(O)CH₃, or —S(O)₂NH₂. Non-limiting examples of substituted aralkyls are: (3-chlorophenyl)-methyl, and 2-chloro-2-phenyl-eth-1-yl.

The term “heteroaryl” when used without the “substituted” modifier refers to a monovalent aromatic group with an aromatic carbon atom or nitrogen atom as the point of attachment, said carbon atom or nitrogen atom forming part of one or more aromatic ring structures wherein at least one of the ring atoms is nitrogen, oxygen or sulfur, and wherein the heteroaryl group consists of no atoms other than carbon, hydrogen, aromatic nitrogen, aromatic oxygen and aromatic sulfur. As used herein, the term does not preclude the presence of one or more alkyl, aryl, and/or aralkyl groups (carbon number limitation permitting) attached to the aromatic ring or aromatic ring system. If more than one ring is present, the rings may be fused or unfused. Non-limiting examples of heteroaryl groups include furanyl, imidazolyl, indolyl, indazolyl (Im), isoxazolyl, methylpyridinyl, oxazolyl, phenylpyridinyl, pyridinyl, pyrrolyl, pyrimidinyl, pyrazinyl, quinolyl, quinazolyl, quinoxalinyl, triazinyl, tetrazolyl, thiazolyl, thienyl, and triazolyl. The term “heteroarenediyl” when used without the “substituted” modifier refers to an divalent aromatic group, with two aromatic carbon atoms, two aromatic nitrogen atoms, or one aromatic carbon atom and one aromatic nitrogen atom as the two points of attachment, said atoms forming part of one or more aromatic ring structure(s) wherein at least one of the ring atoms is nitrogen, oxygen or sulfur, and wherein the divalent group consists of no atoms other than carbon, hydrogen, aromatic nitrogen, aromatic oxygen and aromatic sulfur. As used herein, the term does not preclude the presence of one or more alkyl, aryl, and/or aralkyl groups (carbon number limitation permitting) attached to the aromatic ring or aromatic ring system. If more than one ring is present, the rings may be fused or unfused. Non-limiting examples of heteroarenediyl groups include:

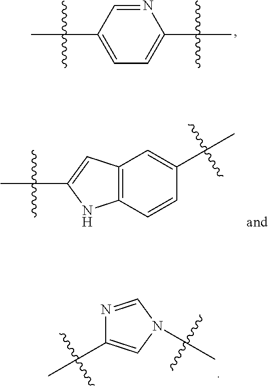 [SMILES: *c1ccc(*)nc1 ; *c1ccc2[nH]c(*)cc2c1 ; *c1cn(*)cn1]

When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —OC(O)CH₃, or —S(O)₂NH₂.

The term “heterocycloalkyl” when used without the “substituted” modifier refers to a monovalent non-aromatic group with a carbon atom or nitrogen atom as the point of attachment, said carbon atom or nitrogen atom forming part of one or more non-aromatic ring structures wherein at least one of the ring atoms is nitrogen, oxygen or sulfur, and wherein the heterocycloalkyl group consists of no atoms other than carbon, hydrogen, nitrogen, oxygen and sulfur. As used herein, the term does not preclude the presence of one or more alkyl groups (carbon number limitation permitting) attached to the ring or ring system. If more than one ring is present, the rings may be fused or unfused. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, and pyranyl. When the term “heterocycloalkyl” used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —OC(O)CH₃, or —S(O)₂NH₂.

The term “acyl” when used without the “substituted” modifier refers to the group —C(O)R, in which R is a hydrogen, alkyl, aryl, aralkyl or heteroaryl, as those terms are defined above. The groups, —CHO, —C(O)CH₃ (acetyl, Ac), —C(O)CH₂CH₃, C(O)CH₂CH₂CH₃, C(O)CH(CH₃)₂, C(O)CH(CH₂)₂, C(O)C₆H₅, C(O)C₆H₄CH₃, —C(O)CH₂C₆H₅, —C(O)(imidazolyl) are non-limiting examples of acyl groups. A “thioacyl” is defined in an analogous manner, except that the oxygen atom of the group —C(O)R has been replaced with a sulfur atom, —C(S)R. When either of these terms are used with the “substituted” modifier one or more hydrogen atom (including the hydrogen atom directly attached the carbonyl or thiocarbonyl group) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —OC(O)CH₃, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples of alkoxy groups include: —OCH₃ (methoxy), —OCH₂CH₃ (ethoxy), —OCH₂CH₂CH₃, —OCH(CH₃)₂ (isopropoxy), —OCH(CH₂)₂, —O-cyclopentyl, and —O-cyclohexyl. The terms “alkenyloxy”, “alkynyloxy”, “aryloxy”, “aralkoxy”, “heteroaryloxy”, and “acyloxy”, when used without the “substituted” modifier, refers to groups, defined as —OR, in which R is alkenyl, alkynyl, aryl, aralkyl, heteroaryl, and acyl, respectively. The term “alkoxydiyl” refers to the divalent group —O-alkanediyl-, —O-alkanediyl-O—, or -alkanediyl-O-alkanediyl-. The term “alkylthio” and “acylthio” when used without the “substituted” modifier refers to the group —SR, in which R is an alkyl and acyl, respectively. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —OC(O)CH₃, or —S(O)₂NH₂. The term “alcohol” corresponds to an alkane, as defined above, wherein at least one of the hydrogen atoms has been replaced with a hydroxy group.

The term “alkylamino” when used without the “substituted” modifier refers to the group —NHR, in which R is an alkyl, as that term is defined above. Non-limiting examples of alkylamino groups include: —NHCH₃ and —NHCH₂CH₃. The term “dialkylamino” when used without the “substituted” modifier refers to the group —NRR′, in which R and R′ can be the same or different alkyl groups, or R and R′ can be taken together to represent an alkanediyl. Non-limiting examples of dialkylamino groups include: —N(CH₃)₂, —N(CH₃)(CH₂CH₃), and N-pyrrolidinyl. The terms “alkoxyamino”, “alkenylamino”, “alkynylamino”, “arylamino”, “aralkylamino”, “heteroarylamino”, and “alkylsulfonylamino” when used without the “substituted” modifier, refers to groups, defined as —NHR, in which R is alkoxy, alkenyl, alkynyl, aryl, aralkyl, heteroaryl, and alkylsulfonyl, respectively. A non-limiting example of an arylamino group is —NHC₆H₅. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. The term “alkylaminodiyl” refers to the divalent group —NH-alkanediyl-, —NH-alkanediyl-NH—, or -alkanediyl-NH-alkanediyl-. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —OC(O)CH₃, or —S(O)₂NH₂. The groups —NHC(O)OCH₃ and —NHC(O)NHCH₃ are non-limiting examples of substituted amido groups.

The term “alkylphosphate” when used without the “substituted” modifier refers to the group —OP(O)(OH)(OR), in which R is an alkyl, as that term is defined above. Non-limiting examples of alkylphosphate groups include: —OP(O)(OH)(OMe) and —OP(O)(OH)(OEt). The term “dialkylphosphate” when used without the “substituted” modifier refers to the group —OP(O)(OR)(OR′), in which R and R′ can be the same or different alkyl groups, or R and R′ can be taken together to represent an alkanediyl. Non-limiting examples of dialkylphosphate groups include: —OP(O)(OMe)₂, —OP(O)(OEt)(OMe) and —OP(O)(OEt)₂. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —OC(O)CH₃, or —S(O)₂NH₂.

The terms “alkylsulfonyl” and “alkylsulfinyl” when used without the “substituted” modifier refers to the groups —S(O)₂R and —S(O)R, respectively, in which R is an alkyl, as that term is defined above. The terms “alkenylsulfonyl”, “alkynylsulfonyl”, “arylsulfonyl”, “aralkylsulfonyl”, and “heteroarylsulfonyl”, are defined in an analogous manner. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —OC(O)CH₃, or —S(O)₂NH₂.

As used herein, a “chiral auxiliary” refers to a removable chiral group that is capable of influencing the stereoselectivity of a reaction. Persons of skill in the art are familiar with such compounds, and many are commercially available.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating a disease, is sufficient to effect such treatment for the disease.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human subjects are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “pharmaceutically acceptable carrier,” as used herein means a pharmaceutically-acceptable material, composition or vehicle, such as a liquid or solid filler, diluent, excipient, solvent or encapsulating material, involved in carrying or transporting a chemical agent.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an inhibitor according to the present invention. The prodrug itself may or may not also have activity with respect to a given target protein. For example, a compound comprising a hydroxy group may be administered as an ester that is converted by hydrolysis in vivo to the hydroxy compound. Suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoate, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, a compound comprising an amine group may be administered as an amide that is converted by hydrolysis in vivo to the amine compound.

A “repeat unit” is the simplest structural entity of certain materials, for example, frameworks and/or polymers, whether organic, inorganic or metal-organic. In the case of a polymer chain, repeat units are linked together successively along the chain, like the beads of a necklace. For example, in polyethylene, —[—CH₂CH₂—]ₙ—, the repeat unit is —CH₂CH₂—. The subscript “n” denotes the degree of polymerization, that is, the number of repeat units linked together. When the value for “n” is left undefined or where “n” is absent, it simply designates repetition of the formula within the brackets as well as the polymeric nature of the material. The concept of a repeat unit applies equally to where the connectivity between the repeat units extends three dimensionally, such as in metal organic frameworks, modified polymers, thermosetting polymers, etc.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2n, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diasteromers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≦15%, more preferably ≦10%, even more preferably ≦5%, or most preferably ≦1% of another stereoisomer(s).

“Substituent convertible to hydrogen in vivo” means any group that is convertible to a hydrogen atom by enzymological or chemical means including, but not limited to, hydrolysis and hydrogenolysis. Examples include hydrolyzable groups, such as acyl groups, groups having an oxycarbonyl group, amino acid residues, peptide residues, o-nitrophenylsulfenyl, trimethylsilyl, tetrahydropyranyl, diphenylphosphinyl, and the like. Examples of acyl groups include formyl, acetyl, trifluoroacetyl, and the like. Examples of groups having an oxycarbonyl group include ethoxycarbonyl, tert-butoxycarbonyl (—C(O)OC(CH₃)₃), benzyloxycarbonyl, p-methoxybenzyloxycarbonyl, vinyloxycarbonyl, β-(p-toluenesulfonyl)ethoxycarbonyl, and the like. Suitable amino acid residues include, but are not limited to, residues of Gly (glycine), Ala (alanine), Arg (arginine), Asn (asparagine), Asp (aspartic acid), Cys (cysteine), Glu (glutamic acid), His (histidine), Ile (isoleucine), Leu (leucine), Lys (lysine), Met (methionine), Phe (phenylalanine), Pro (proline), Ser (serine), Thr (threonine), Trp (tryptophan), Tyr (tyrosine), Val (valine), Nva (norvaline), Hse (homoserine), 4-Hyp (4-hydroxyproline), 5-Hyl (5-hydroxylysine), Orn (ornithine) and β-Ala. Examples of suitable amino acid residues also include amino acid residues that are protected with a protecting group. Examples of suitable protecting groups include those typically employed in peptide synthesis, including acyl groups (such as formyl and acetyl), arylmethoxycarbonyl groups (such as benzyloxycarbonyl and p-nitrobenzyloxycarbonyl), tert-butoxycarbonyl groups (—C(O)OC(CH₃)₃), and the like. Suitable peptide residues include peptide residues comprising two to five amino acid residues. The residues of these amino acids or peptides can be present in stereochemical configurations of the D-form, the L-form or mixtures thereof. In addition, the amino acid or peptide residue may have an asymmetric carbon atom. Examples of suitable amino acid residues having an asymmetric carbon atom include residues of Ala, Leu, Phe, Trp, Nva, Val, Met, Ser, Lys, Thr and Tyr. Peptide residues having an asymmetric carbon atom include peptide residues having one or more constituent amino acid residues having an asymmetric carbon atom. Examples of suitable amino acid protecting groups include those typically employed in peptide synthesis, including acyl groups (such as formyl and acetyl), arylmethoxycarbonyl groups (such as benzyloxycarbonyl and p-nitrobenzyloxycarbonyl), tert-butoxycarbonyl groups (—C(O)OC(CH₃)₃), and the like. Other examples of substituents “convertible to hydrogen in vivo” include reductively eliminable hydrogenolyzable groups. Examples of suitable reductively eliminable hydrogenolyzable groups include, but are not limited to, arylsulfonyl groups (such as o-toluenesulfonyl); methyl groups substituted with phenyl or benzyloxy (such as benzyl, trityl and benzyloxymethyl); arylmethoxycarbonyl groups (such as benzyloxycarbonyl and o-methoxy-benzyloxycarbonyl); and haloethoxycarbonyl groups (such as β,β,β-trichloroethoxycarbonyl and β-iodoethoxycarbonyl).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

Modifications or derivatives of the compounds, agents, and active ingredients disclosed throughout this specification are contemplated as being useful with the methods and compositions of the present invention. Derivatives may be prepared and the properties of such derivatives may be assayed for their desired properties by any method known to those of skill in the art, such as methods described herein.

In certain aspects, “derivative” refers to a chemically-modified compound that still retains the desired effects of the compound prior to the chemical modification. A “Wnt protein signalling inhibitor derivative,” therefore, refers to a chemically modified Wnt protein signalling inhibitor that still retains the desired effects of the parent Wnt protein signalling inhibitor prior to its chemical modification. Such effects may be enhanced (e.g., slightly more effective, twice as effective, etc.) or diminished (e.g., slightly less effective, 2-fold less effective, etc.) relative to the parent Wnt protein signalling inhibitor, but may still be considered a Wnt protein signalling inhibitor derivative. Such derivatives may have the addition, removal, or substitution of one or more chemical moieties on the parent molecule. Non-limiting examples of the types of modifications that can be made to the compounds and structures disclosed herein include the addition or removal of lower unsubstituted alkyls such as methyl, ethyl, propyl, or substituted lower alkyls such as hydroxymethyl or aminomethyl groups; carboxyl groups and carbonyl groups; hydroxyls; nitro, amino, amide, imide, and azo groups; sulfate, sulfonate, sulfono, sulfhydryl, sulfenyl, sulfonyl, sulfoxido, sulfonamide, phosphate, phosphono, phosphoryl groups, and halide substituents. Additional modifications can include an addition or a deletion of one or more atoms of the atomic framework, for example, substitution of an ethyl by a propyl, or substitution of a phenyl by a larger or smaller aromatic group. Alternatively, in a cyclic or bicyclic structure, heteroatoms such as N, S, or O can be substituted into the structure instead of a carbon atom.

Prodrugs and solvates of the compounds of the present invention are also contemplated herein. The term “prodrug,” as used herein, is understood as being a compound which, upon administration to a subject, such as a mammal, undergoes chemical conversion by metabolic or chemical processes to yield a compound any of the formulas herein, or a salt and/or solvate thereof. Solvates of the compounds of the present invention are preferably hydrates.

As used herein, “protecting group” refers to a moiety attached to a functional group to prevent an otherwise unwanted reaction of that functional group. The term “functional group” generally refers to how persons of skill in the art classify chemically reactive groups. Examples of functional groups include hydroxyl, amine, sulfhydryl, amide, carboxyl, carbonyl, etc. Protecting groups are well-known to those of skill in the art. Non-limiting exemplary protecting groups fall into categories such as hydroxy protecting groups, amino protecting groups, sulfhydryl protecting groups and carbonyl protecting groups. Such protecting groups may be found in Greene and Wuts, 1999, incorporated herein by reference in its entirety. The Wnt protein signalling inhibitors described herein are also contemplated as protected by one or more protecting groups—that is, the inhibitors are contemplated in their “protected form.”

Compounds of the present invention may contain one or more asymmetric centers and thus can occur as racemates and racemic mixtures, single enantiomers, diastereomeric mixtures and individual diastereomers. In certain embodiments, a single diastereomer is present. All possible stereoisomers of the compounds of the present invention are contemplated as being within the scope of the present invention. However, in certain aspects, particular diastereomers are contemplated. The chiral centers of the compounds of the present invention can have the S- or the R-configuration, as defined by the IUPAC 1974 Recommendations. In certain aspects, certain compounds of the present invention may comprise S- or R-configurations at particular carbon centers.

Synthetic techniques that may be used to prepare certain compounds of the present invention are provided in the Examples section. Other synthetic techniques to prepare compounds of the present invention as well as derivatives are well-known to those of skill in the art. For example, Smith and March, 2001 discuss a wide variety of synthetic transformations, reaction conditions, and possible pitfalls relating thereto. Methods discussed therein may be adapted to prepare compounds of the present invention from commerically available starting materials.

Solvent choices for preparing compounds of the present invention will be known to one of ordinary skill in the art. Solvent choices may depend, for example, on which one(s) will facilitate the solubilizing of all the reagents or, for example, which one(s) will best facilitate the desired reaction (particularly when the mechanism of the reaction is known). Solvents may include, for example, polar solvents and non-polar solvents. Solvents choices include, but are not limited to, tetrahydrofuran, dimethylformamide, dimethylsulfoxide, dioxane, methanol, ethanol, hexane, methylene chloride and acetonitrile. More than one solvent may be chosen for any particular reaction or purification procedure. Water may also be admixed into any solvent choice. Further, water, such as distilled water, may constitute the reaction medium instead of a solvent.

Persons of ordinary skill in the art will be familiar with methods of purifying compounds of the present invention. One of ordinary skill in the art will understand that compounds of the present invention can generally be purified at any step, including the purification of intermediates as well as purification of the final products. In preferred embodiments, purification is performed via silica gel column chromatography or HPLC.

In view of the above definitions, other chemical terms used throughout this application can be easily understood by those of skill in the art. Terms may be used alone or in any combination thereof.

The above definitions supersede any conflicting definition in any of the reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### V. PHARMACEUTICAL FORMULATIONS AND ROUTES FOR ADMINISTRATION

Pharmaceutical compositions of the present invention comprise an effective amount of one or more candidate substances (e.g., a Wnt protein signalling inhibitor) or additional agents dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one candidate substance or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, pp 1289-1329, 1990). Except insofar as any conventional carrier is incompatible with the active ingredient, its use in the therapeutic or pharmaceutical compositions is contemplated.

The candidate substance may comprise different types of carriers depending on whether it is to be administered in solid, liquid or aerosol form, and whether it needs to be sterile for such routes of administration as injection. Compounds of the present invention may be administered orally, intraadiposally, intraarterially, intraarticularly, intracranially, intradermally, intralesionally, intramuscularly, intranasally, intraocularally, intrapericardially, intraperitoneally, intrapleurally, intraprostaticaly, intrarectally, intrathecally, intratracheally, intratumorally, intraumbilically, intravaginally, intravenously, intravesicularlly, intravitreally, liposomally, locally, mucosally, orally, parenterally, rectally, subconjunctival, subcutaneously, sublingually, topically, transbuccally, transdermally, vaginally, in crèmes, in lipid compositions, via a catheter, via a lavage, via continuous infusion, via infusion, via inhalation, via injection, via local delivery, via localized perfusion, bathing target cells directly, or by other method or any combination of the foregoing as would be known to one of ordinary skill in the art (see, for example, Remington's Pharmaceutical Sciences, 1990). In particular embodiments, the composition may be formulated for oral delivery. Pharmaceutical compositions comprising a compound of the present invention are also contemplated, and such compositions may be adapted for administration via any method known to those of skill in the art, such as the methods described above.

In particular embodiments, the composition is administered to a subject using a drug delivery device. Any drug delivery device is contemplated for use in delivering a pharmaceutically effective amount of a Wnt protein signalling inhibitor.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will typically determine the concentration of active ingredient(s) in a composition and appropriate dose(s) for the individual subject.

The dose can be repeated as needed as determined by those of ordinary skill in the art. Thus, in some embodiments of the methods set forth herein, a single dose is contemplated. In other embodiments, two or more doses are contemplated. Where more than one dose is administered to a subject, the time interval between doses can be any time interval as determined by those of ordinary skill in the art. For example, the time interval between doses may be about 1 hour to about 2 hours, about 2 hours to about 6 hours, about 6 hours to about 10 hours, about 10 hours to about 24 hours, about 1 day to about 2 days, about 1 week to about 2 weeks, or longer, or any time interval derivable within any of these recited ranges.

In certain embodiments, it may be desirable to provide a continuous supply of a pharmaceutical composition to the patient. This could be accomplished by catheterization, followed by continuous administration of the therapeutic agent, for example. The administration could be intra-operative or post-operative.

In certain embodiments, pharmaceutical compositions may comprise, for example, at least about 0.1% of a Wnt protein signalling inhibitor. In other embodiments, the Wnt protein signalling inhibitor may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal, or combinations thereof.

The Wnt protein signalling inhibitor may be formulated into a composition, such as a pharmaceutical composition, in a free base, neutral, or salt form. Pharmaceutically acceptable salts are described herein.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. It may be preferable to include isotonic agents, such as, for example, sugars, sodium chloride, or combinations thereof.

In other embodiments, one may use eye drops, nasal solutions or sprays, aerosols or inhalants in the present invention. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in certain embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments the candidate substance is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. In certain embodiments, carriers for oral administration comprise inert diluents (e.g., glucose, lactose, or mannitol), assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain embodiments an oral composition may comprise one or more binders, excipients, disintegration agents, lubricants, flavoring agents, or combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar, or both.

Sterile injectable solutions may be prepared by incorporating a compound of the present invention in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, certain methods of preparation may include vacuum-drying or freeze-drying techniques which yield a powder of the active ingredient plus any additional desired ingredient from a previously sterile-filtered liquid medium thereof. The liquid medium should be suitably buffered if necessary and the liquid diluent (e.g., water) first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition should be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin, or combinations thereof.

### VI. COMBINATION THERAPY

In order to enhance or increase the effectiveness of a Wnt protein signalling inhibitor of the present invention, the inhibitor may be combined with another therapy, such as another agent that combats and/or prevents cancer, osteopetrosis, a degenerative disease, or type II diabetes. For example, Wnt protein signalling inhibitors of the present invention may be provided in a combined amount with an effective amount another agent that is known to reduce tumor size.

It is contemplated that combination therapy of the present invention may be used in vitro or in vivo. These processes may involve administering the agents at the same time or within a period of time wherein separate administration of the substances produces a desired therapeutic benefit. This may be achieved by contacting the cell, tissue, or organism with a single composition or pharmacological formulation that includes two or more agents, or by contacting the cell with two or more distinct compositions or formulations, wherein one composition includes one agent and the other includes another.

The compounds of the present invention may precede, be co-current with and/or follow the other agents by intervals ranging from minutes to weeks. In embodiments where the agents are applied separately to a cell, tissue or organism, one would generally ensure that a significant period of time did not expire between the time of each delivery, such that the agents would still be able to exert an advantageously combined effect on the cell, tissue or organism. For example, in such instances, it is contemplated that one may contact the cell, tissue or organism with two, three, four or more modalities substantially simultaneously (i.e., within less than about a minute) as the candidate substance. In other aspects, one or more agents may be administered about 1 minute, about 5 minutes, about 10 minutes, about 20 minutes about 30 minutes, about 45 minutes, about 60 minutes, about 2 hours, about 3 hours, about 4 hours, about 5 hours, about 6 hours, about 7 hours about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 1, about 2, about 3, about 4, about 5, about 6, about 7 or about 8 weeks or more, and any range derivable therein, prior to and/or after administering the candidate substance.

Various combination regimens of the agents may be employed. Non-limiting examples of such combinations are shown below, wherein a Wnt protein signalling inhibitor is “A” and a second agent, such as an anti-cancer agent, is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

A. Anti-Cancer Therapy

An anti-cancer agent may be used in combination therapy with Wnt protein signalling inhibitors of the present invention. As used herein, an “anti-cancer” agent is capable of negatively affecting cancer in a subject, for example, by killing one or more cancer cells, inducing apoptosis in one or more cancer cells, reducing the growth rate of one or more cancer cells, reducing the incidence or number of metastases, reducing tumor size, inhibiting tumor growth, reducing the blood supply to a tumor or one or more cancer cells, promoting an immune response against one or more cancer cells or a tumor, preventing or inhibiting the progression of a cancer, or increasing the lifespan of a subject with a cancer. Anti-cancer agents are well-known in the art and include, for example, chemotherapy agents (chemotherapy), radiotherapy agents (radiotherapy), a surgical procedure, immune therapy agents (immunotherapy), genetic therapy agents (gene therapy), reoviral therapy, hormonal therapy, other biological agents (biotherapy), and/or alternative therapies.

B. Osteopetrosis Therapy

Osteopetrosis, also known as marble bone disease and Albers-Schonberg disease, is an extremely rare inherited disorder whereby the bones harden, becoming denser, in contrast to the more prevalent osteomalacia, in which the bones soften. Bone marrow transplant therapy may be combined with administration of Wnt protein signalling inhibitors of the present invention to treat or prevent osteopetrosis. Other treatments targeting osteopetrosis that may be combined with Wnt protein signalling inhibitors described herein include those disclosed in the following documents, each of which is incorporated herein by reference: U.S. Pat. Nos. 7,241,732; 7,186,683; 6,943,151; 6,833,354; 6,699,873; 6,686,148; 5,806,529; 5,777,193; RE35,694; 5,641,747; and 4,843,063.

C. Degenerative Disease Therapy

As discussed herein, degenerative diseases may be treated using Wnt protein signalling inhibitors of the present invention. Accordingly, other treatments that target degenerative diseases may be combined with administration of the Wnt protein signalling inhibitors. Non-limiting examples of degenerative diseases include type II diabetes and age-related impairment of tissue repair.

1. Type II Diabetes Therapy

Type II diabetes is a chronic, progressive disease that has no clearly established cure. It is a metabolic disorder that is primarily characterized by insulin resistance, relative insulin deficiency and hyperglycemia. Treatment options that may be combined with Wnt protein signalling inhibitor administration include exercise, diet management to control the intake of glucose, and use of anti-diabetic drugs (e.g., metformin, phenformin, repaglinide, nateglinide, rosiglitazone, pioglitazone or miglitol).

2. Age-Related Impairment of Tissue Repair Therapy

A variety of tissues degenerate over time as one ages, such as skeletal muscle and organ tissues (e.g., heart, kidney, lung and liver). Wnt protein signalling inhibition has been implicated in, for example, muscle regeneration (Brack et al., 2007). Therapies pertaining to age-related impairment of tissue repair that may be combined with Wnt protein signalling inhibitor administration include, for example, gene therapy, such as described by Barton-Davis et al. (1998; incorporated herein by reference) and drugs described by Lynch (2004; incorporated herein by reference).

### VII. EXAMPLES

The following examples are included to demonstrate certain preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Reagents. Antibodies purchased from the following sources: Santa Cruz Biotechnology (Myc-9E10), Bethyl Laboratories (Human IgGFc), Cell Signaling Technology (Dv12, Lrp6-C5C7, pJnk Thr183/Tyr185), and Sigma (Kif3a). The University of Texas Southwestern Medical Center chemical library is assembled from ChemDiv, ChemBridge, ComGenex, Prestwick, and TimT3k collections. C16 ω-alkynyl fatty acid (alkynyl-PA) was synthesized as previously described (Gao et al., 2011). Biotin-azide and buffers required for click chemistry were purchased from Invitrogen. Membrane fractionation buffer made from 10 mM HEPES, 10 mM KCl, 1.5 mM MgCl₂, 1 mM Na-EDTA, and 250 mM sucrose in water, pH 7.4 Membrane solubilization buffer consisted of 100 mM MES, 20 mM NaCl, 1 mM DTT, 0.2 mM EDTA, 0.05% TX-100, 0.2% glycerol and 0.15% octylglucoside, pH 6.5. PL buffer contained 10 mM Tris-HCl, 150 mM NaCl, pH 7.5. pCMV-GLuc control plasmid from New England Biolabs. Hhat and Goat constructs were a generous gift from Mike Brown and Joe Goldstein. To generate Gaussia luciferase (GL) fusion proteins, GL lacking its signaling sequence was cloned into pcDNA3.1 and then cDNAs encoding various Wnt proteins subsequently ligated in frame. PCR-based site directed mutagenesis was used to generate Porcn H335L.

Luciferase reporter assays. Wnt-Gaussia luciferase secretion and SuperTopFlash assays were conducted as described using a Dual Luciferase kit (Promega) (Chen et al., 2009).

Flow cytometry. The indicated constructs were introduced into Cos1 or HEK293 cells via Fugene6 transfection (Roche), 6 well format, and expressed for 48 hours. New media containing 100 nM IWP-Cy3 and an IWP (15 mM) or DMSO was added for 12 hours. Following 3xPBS washes, cells were trypsinized, pelleted, resuspended in cold PBS, and kept on ice. The gate for IWP-Cy3 positive cells was defined as the region excluding the bulk population in cells transfected with control DNA. The gate for IWP-Cy3 cells was defined as the region excluding the bulk population of cells labeled in control DNA transfected cells. Cells diverging from the SSC/FSC primary population were excluded from analysis. Flow cytometry was carried out with a FACS Calibur (BD Biosciences) and data analyzed on Cell Quest Pro (BD Biosciences).

Click chemistry. HEK293 cells transiently transfected with the Wnt3A-Fc DNA expression construct were treated with C16 ω-alkynyl fatty acid (see Reagents; 100 μM final concentration) for six hours as previously described (9) in the presence or DMSO or various IWP compounds. C16 ω-alkynyl fatty acid-labeled Wnt3A-Fc protein isolated from cell lysate using Protein A sepharose was then subjected to a copper catalyzed alkyne-azide cycloaddition with biotin-azide with protein immobilized on the sepharose. The biotinylated Wnt protein run on SDS PAGE was detected using HRP conjugated streptavidin.

Organotypic kidney culture. E11.5 urogenital systems were removed and bisected in sterile phosphate buffered saline (PBS) and then the individual halves were cultured in 350 mL of media at the air-media interface on 24-well tissue culture treated, 6.5 mm diameter, 8.0 mM pore size Transwell filters (Corning, catalog no. 3422). The media (DMEM with 10% fetal bovine serum (FBS) and Pen/Strep) was supplemented with either DMSO or IWP2 and replaced with fresh media every 12, 24, or 48 hours. All treatments were repeated at least three times with a minimum of six individual kidneys from six distinct embryos each time.

Zebrafish studies. All zebrafish experiments were performed in accordance to regulatory standards as accepted by the Institutional Animal Care and Use Committee (IACUC) at the University of Texas Southwestern Medical Center. To determine a comparable concentration of IWP12 and IWR1 in zebrafish, 7X TCFsiam:EGFP embryos at 4 hours after fertilization, expressing EGFP under the control of seven TCF binding elements and a siamois minimal promoter were incubated with E3 medium (5 mM NaCl, 0.17 mM KCl, 0.33 mM CaCl₂, 0.33 mM MgSO₄) containing DMSO, IWR1, IWP12, and/or GSK3β inhibitor 1 (Calbiochem) for 20 hours and subsequently EGFP signals quantified by measuring pixel density from the embryo pictures. Three different pictures of the embryos were taken and the pixel numbers were measured by ImageJ software. For caudal fin regeneration assay, zebrafish larvae at 3 days after fertilization were anaesthetized in 0.02% (v/v) Tricaine, and half of the fins resected using a razor blade. Subsequently, the larvae were reared at 28 C in E3 medium containing DMSO or IWR1 (10 μM) or IWP-12 (50 μM) for an additional 4 days. Wholemount in situ hybridization was performed at 10 hours after fertilization with digoxigenin-labeled antisense RNA probes generated against d1x3b, nt1, and ctsl1b/hgg1. Whole-mount in situ hybridization was performed at 24 hours after fertilization with eng1a. Primers used for generating in situ probes:

dlx3b forward:
(SEQ ID NO: 1)
5′- CAACA GAGGGAGTGTGAGAAAGC
dlx3b reverse:
(SEQ ID NO: 2)
5′- AACCTCGCCGTTCTTGTAAAGC
ntl forward:
(SEQ ID NO: 3)
5′- GAATGAAGAGATTACCGCTCTG
ntl reverse:
(SEQ ID NO: 4)
5′- CCAAGATCAAGTCCA TAACTGC
ctsl1b/(hgg1) forward:
(SEQ ID NO: 5)
5′- TGATGTTTGCTTTGCTCGTCAC
ctsl1b/(hgg1) reverse:
(SEQ ID NO: 6)
5′- GAACTGTAGGGATTGATGTGATGC
eng1a forward:
(SEQ ID NO: 7)
5′- GGAGGGCAGGACTGATCTCTG
eng1a reverse:
(SEQ ID NO: 8)
5′- GCGTAATATAGGCTACAACACC

Zebrafish embryonic cell cultures were initiated from embryos at the shield stage (6 hpf). The embryos were dissociated in trypsin/EDTA solution with gentle homogenization and pipetting. After centrifugation, the collected cells were resuspended in F12/L15/DMEM medium and placed into a 24-well tissue culture plate.

### Example 2

### Results

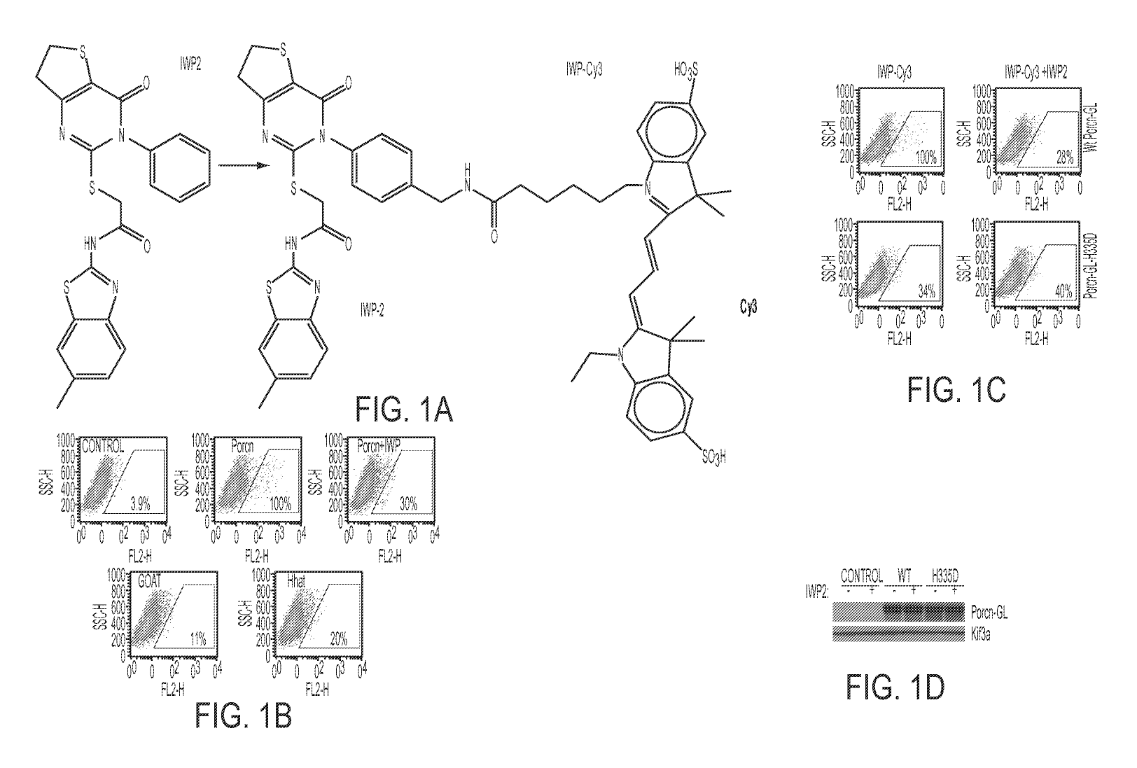

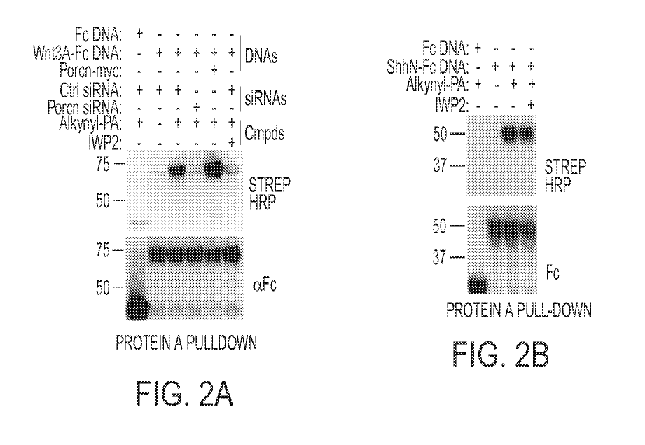

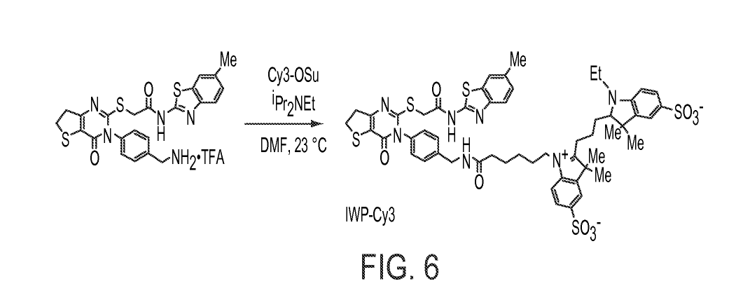

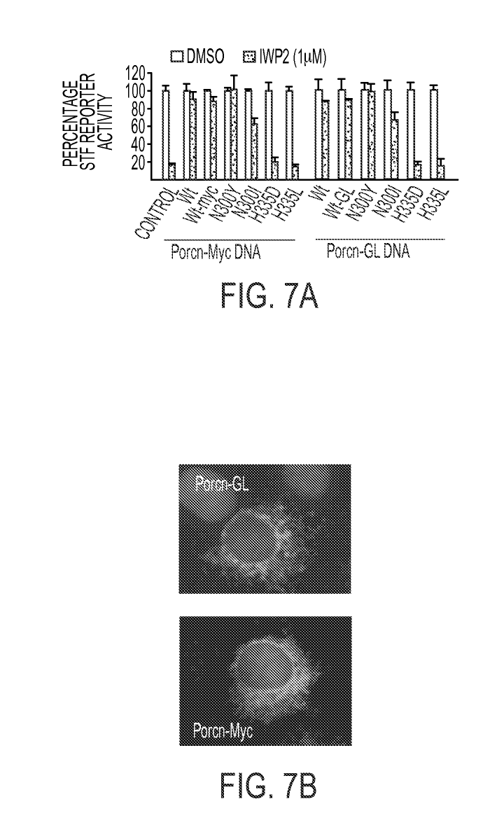

To better understand the interaction between IWP compounds and Porcn, the inventors generated a fluorescently labeled reagent based on the IWP2 molecule (IWP-Cy3; FIG. 1A; FIG. 6) that enabled detection of IWP compound association with Porcn-expressing cells (FIG. 1B). Cells expressing Porcn-related MBOAT family members (Goat or Hhat) were not labeled with IWP-Cy3 suggesting a direct interaction between IWP2 and Porcn. Consistent with this model, an inactive Porcn protein harboring a mutation in a highly conserved and presumed active-site residue was unable to bind to IWP-Cy3 (FIGS. 1C-D; FIGS. 7A-B) (Barrott et al., 2011; Galli et al., 2007). Using click chemistry technology, the inventors confirmed that IWP2 disrupts Wnt protein acylation (FIG. 2A). They also demonstrated that 3 IWP2 does not block fatty acylation of the related Hh signaling molecule mediated by Hhat, another MBOAT family member (FIG. 2B). Furthermore, the inventors have previously demonstrated that IWP2 does not block general protein secretion or cellular responses mediated by the Hh and Notch proteins (Chen et al., 2009).

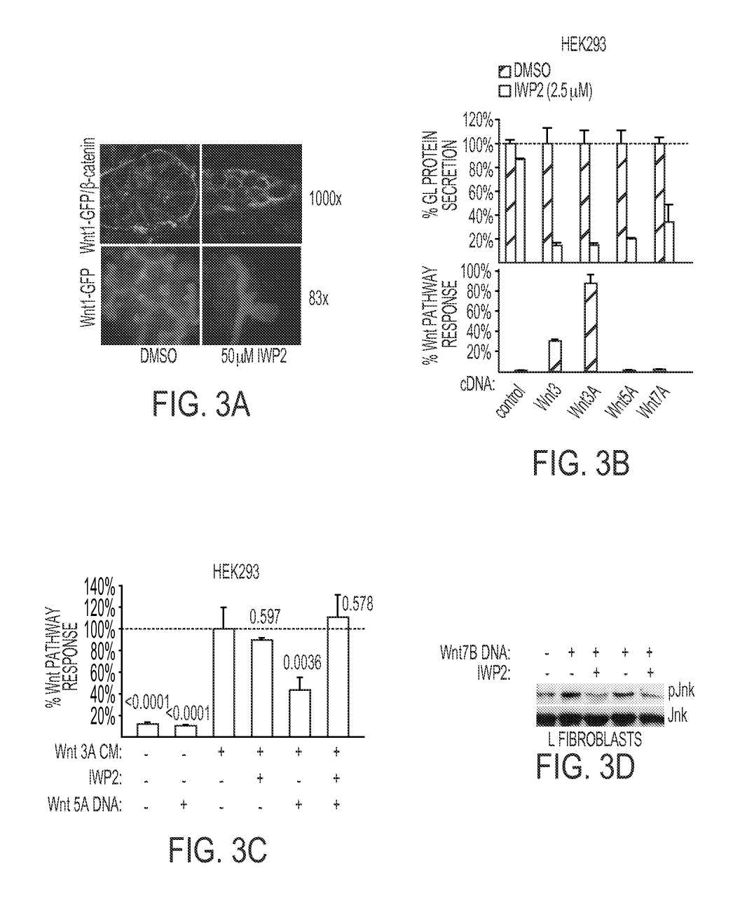

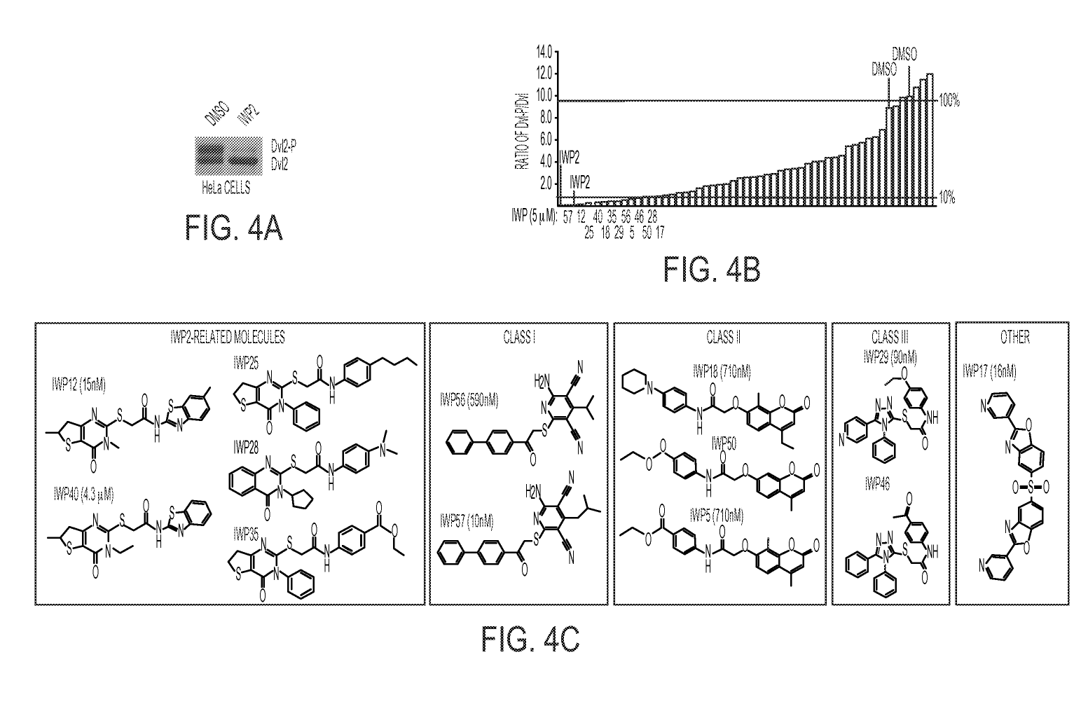

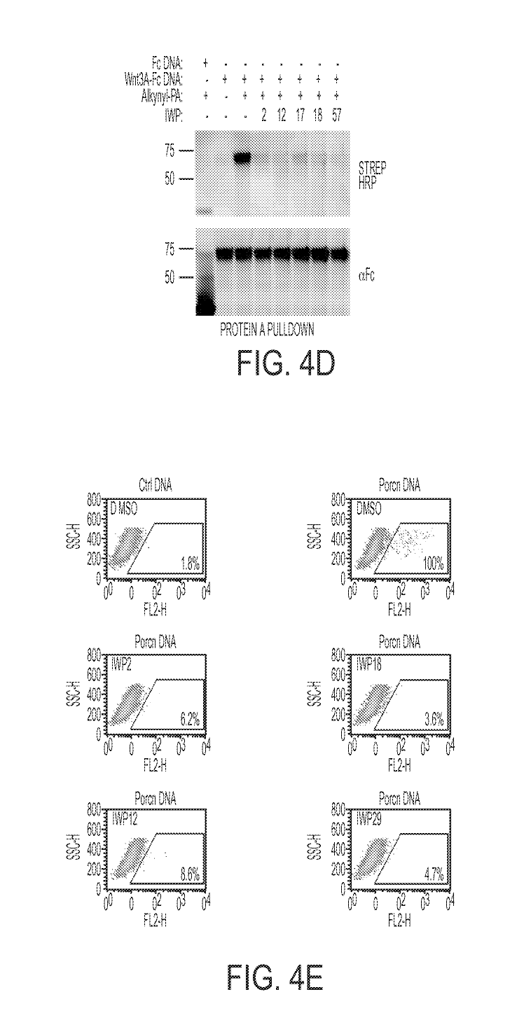

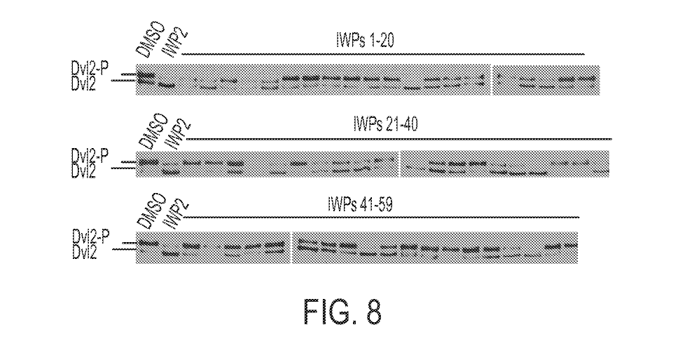

The transport of Wnt proteins through the secretory pathway relies upon the chaperone protein Wnt1ess (W1s) which binds only to Wnt proteins lipidated on a conserved serine residue (Coombs et al., 2010; Herr and Basler, 2012). Using in vitro cultured embryonic kidney tissue derived from Wnt1-GFP expressing transgenic mice, the inventors demonstrated that IWP2 can block the accumulation of Wnt1 on the cell surface and concomitantly disrupt tubule induction, a Wnt/β-catenin-dependent process (Merkel et al., 2007) (FIG. 3A). The addition of IWP2 to cells expressing one of several Wnt proteins, including those unable to elicit Wnt/β-catenin pathway response, abrogated the accumulation of Wnt proteins in the cell culture medium consistent with a general role for Porcn in the production of Wnt proteins (FIG. 3B). The inventors demonstrated that this blockade in protein maturation correlates with loss of non-canonical Wnt activity using an assay that measures Wnt5a-dependent antagonism of canonical Wnt pathway response (Lee et al., 2010) (FIG. 3C). Additionally, activation of the non-canonical Wnt pathway effector Jnk in fibroblasts is disrupted by IWP2 (FIG. 3D). Taken together, these observations support a general role for lipidation in the maturation of Wnt family members and the utility of IWP2 for interrogating diverse forms of Wnt-mediated cellular responses. The same chemical library screen that yielded IWP2 also uncovered ˜50 other molecules with potential activity against Wnt protein production (Chen et al., 2009). In addition to previous studies demonstrating that all of these compounds exhibit activity for Wnt- but not Hh- or Notch-dependent signaling (Chen et al., 2009), the inventors biochemically validated the Wnt-inhibitory activity of these chemicals in HeLa cells that exhibit elevated levels of cell autonomous Wnt signaling (Jacob et al., 2011) (FIG. 4A). With the exception of five compounds, all other putative Wnt inhibitors blocked Wnt-induced phosphorylation of Dv1, a signaling molecule directly activated by the Frizzled family of Wnt receptors (Gao and Chen, 2010) (FIG. 4B; FIG. 8).

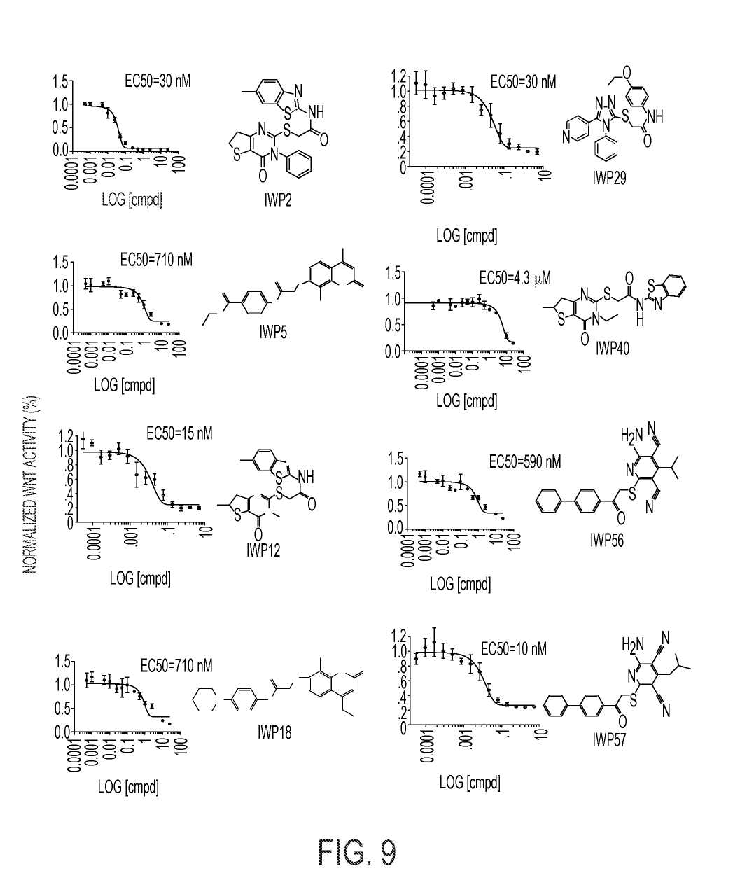

Organizing the top twelve compounds based upon their similarity to IWP2 (or otherwise shared chemical scaffolds) revealed four distinct chemical classes capable of specifically inhibiting Wnt/β-catenin transcriptional response (see Chen et al., 2009) by targeting a component functioning upstream of Dv1, presumably at the level of Wnt protein production (FIG. 4C; FIG. 9). Representative molecules from the different classes, which are structurally distinct from IWP2 class compounds, likely function as Porcn inhibitors given their ability to inhibit Wnt fatty acylation as determined using the click chemistry strategy, and to compete with IWP-Cy3 binding for Porcn (FIGS. 4D-E).

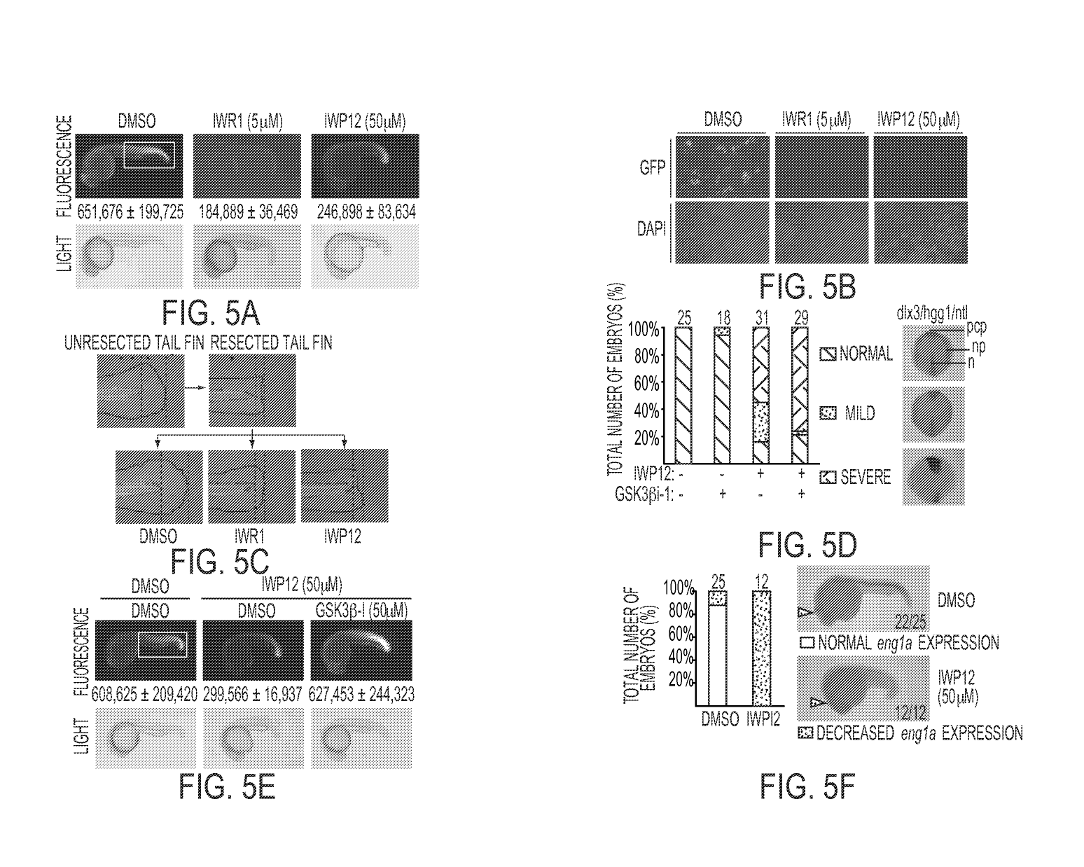

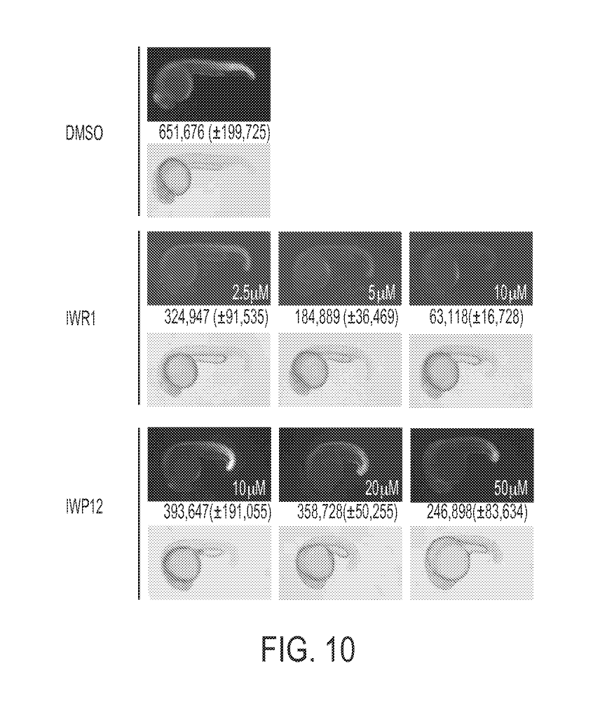

Thus, these diverse chemical structures likely engage the same protein pocket in Porcn to disrupt its activity. Despite earlier successes in achieving chemical targeting of Wnt/β-catenin signaling in zebrafish using the IWR class of Tankyrase inhibitors (Chen et al., 2009; Huang et al., 2009), the inventors were previously unable to demonstrate similar activity using Porcn inhibitors, possibly as a result of poor bioavailability. Evaluating the ability of several new IWP compounds to inhibit in vivo Wnt-mediated response using a transgenic zebrafish line harboring a Wnt-responsive GFP reporter (7XTCF-siam:EGFP; E. Moro et al., unpublished), the inventors uncovered a loss of Wnt signaling activity in animals treated with IWP12 (FIG. 5A; FIG. 10). Cultured embryonic fibroblasts from the same transgenic line also revealed loss of Wnt/β-catenin pathway responses when treated with an IWR compound or IWP12 (FIG. 5B).

Accordingly, IWP12 was further able to block juvenile fish tailfin regeneration following resection, a Wnt/β-catenin pathway-dependent process (Chen et al., 2009; Stoick-Cooper et al., 2007) (FIG. 5C). The weaker Wnt/β-catenin signaling inhibitory activity observed with IWP12 as compared to IWR1 was nevertheless associated with a severe effect on posterior body morphogenesis, possibly signifying additional effects of Porcn disruption on non-canonical Wnt signaling (Marlow et al., 2004) (see FIG. 5A). Whereas the role of Wnt lipidation during Wnt/β-catenin signaling is well validated, its contribution to β-catenin-independent Wnt cellular responses is unclear (Biechele et al., 2011). Based on the in vitro and in vivo results, the inventors anticipated that IWP12 may be useful for studying these other forms of Wnt signaling in vivo. Indeed, IWP 12 was able to block convergence and extension gastrulation movements, a process dependent upon Wnt-planar cell polarity (Wnt/PCP) signaling (Roszko et al., 2009; Sepich et al., 2011) (FIG. 5D). This defect was not rescued by the addition of a Gsk3β inhibitor (Gsk3β inhibitor 1 or Gsk3βi-1), amolecule that blocks 3-catenin destruction and reverses the effects of IWP12 on Wnt/β-catenin pathway activity (FIG. 5E). These observations taken together are consistent with a biosynthetic role for Wnt protein lipidation and β-cateninindependent Wnt-mediated development processes.

In addition to the complexities of phenotypic analysis associated with overlapping roles of various genes in β-catenin-dependent and -independent Wnt signaling, the presence of multiple Wnt proteins in vertebrates has limited the ability to recognize Wnt functions in developmental processes. The inventors demonstrated the utility of a chemically based approach to reveal a role for Wnt-dependent signaling in midbrain/hindbrain boundary (MHB) formation, a process previously shown to be coordinated by three Wnt proteins with overlapping functions—Wnt1, Wnt3A, and Wnt10B (Buckles et al., 2004). Similar to animals lacking all three genes that fail to develop the MHB constriction, embryos treated with IWP12 exhibited decreased expression of the MHB marker Engrailed (eng1a) (Ekker et al., 1992) (FIG. 5F). The inventors anticipate that additional functions of this large family of signaling molecules in vertebrate development could be readily uncovered by limiting the influence of gene redundancy on phenotypic outcome using this chemically based strategy.

### Example 3

### Discussion

The present study reveals Porcn to be a chemical vulnerability in multiple Wnt signaling processes including those governing β-catenin-independent events such as Wnt/PCP signaling. This vulnerability forms the basis of a chemical strategy described herein for probing the participation of different forms of Wnt signaling in vivo. These Porcn inhibitors combined with Tnks and Gsk3β antagonists should facilitate the systematic identification of Wnt-dependent cellular processes in vertebrate embryogenesis and tissue regeneration not readily achievable with classical genetic approaches.

The shared sensitivity of Wnt proteins that mediate different cellular responses to Porcn inhibitors are consistent with previous findings that implicate Porcn activity to be essential to the production of most if not all Wnt proteins (Bartscherer and Boutros, 2008; Port and Basler, 2010). Furthermore, this observation suggests that the Wnt chaperone Wls, which binds to fatty acylated Wnt proteins, is similarly required for the production of most if not all Wnt proteins. Yet, the dependence of individual Wnt activities upon Porcn may vary as a consequence of differences in: a) the ligand dose required to engage cellular responses, b) the determinants that promote ER retention of non-acylated Wnt proteins, and c) the participation of other acyltransferases that modify Wnt proteins. These possibilities may in part contribute to previously observed differences in assignment of Porcn function to various Wnt-dependent processes (Barrott et al., 2011; Biechele et al., 2011; Chen et al., 2012). Evidence that at least some Wnt proteins may also harbor monounsaturated fatty acid modifications suggests that specific Wnt functions may also be dictated by a complex fatty acyl code that could be better understood with the aid of the chemical tools described here (Takada et al., 2006; Mulligan et al., 2012).

Porcn exhibits an ability to accommodate diverse chemical inhibitors, potentially indicating an abundance of opportunities for the refinement of IWP compounds as chemical probes and therapeutic agents. Given that crystallographically guided clinical development of small molecules will not likely be forthcoming for Porcn inhibitors due to the polytopic nature of Porcn, the chemical portfolio described here should improve the understanding of how these molecules achieve Porcn inhibitory activity and how they can be evolved for clinical use. Porcn is a founding member of a large protein family with roles in the production of other important signaling molecules such as the cell-fate determination molecule Hedgehog and the appetite-controlling hormone Ghrelin (Yang et al., 2008; Buglino and Resh, (2008). Thus, these findings should also facilitate the development of small molecules targeting other important signaling processes relevant to disease.

All of the methods and apparatuses disclosed and claimed herein can be made and executed without undue experimentation in light of the present disclosure. While the compositions and methods of this invention have been described in terms of preferred embodiments, it will be apparent to those of skill in the art that variations may be applied to the methods and apparatuses and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VIII. REFERENCES

The following references, to the extent that they provide exemplary procedural or other details supplementary to those set forth herein, are specifically incorporated herein by reference.
U.S. Pat. No. 4,843,063
U.S. Pat. No. 5,641,747
U.S. Pat. No. 5,777,193
U.S. Pat. No. 5,806,529
U.S. Pat. No. 6,686,148
U.S. Pat. No. 6,699,873
U.S. Pat. No. 6,833,354
U.S. Pat. No. 6,943,151
U.S. Pat. No. 7,186,683
U.S. Pat. No. 7,241,732
U.S. Pat. RE35,694
Ailles and Weissman, Curr. Opin. Biotech., 18:460-466, 2007.
Angers and Moon, Nature Rev., 10:468-477, 2009.
Barker and Clevers, Nat. Rev. Drug Discov., 5:997-1014, 2006.
Barrott et al., Proc. Natl. Acad. Sci. USA, 108:12752- 12757, 2011.
Barton-Davis et al., Proc. Natl. Acad. Sci. USA, 95:15603, 1998.
Bartscherer and Boutros, EMBO Rpts., 9:977-982, 2008.
Biechele et al., Dev. Biol., 355:275-285, 2011.
Brack et al., Science, 317:807-810, 2007.
Buckles et al., Mech. Dev., 121:437-447, 2004.
Buglino and Resh, J. Biological Chem., 283:22076-22088, 2008.
Chen et al., J. Cell Sci. 2012 (Epub ahead of print)
Chen et al., Nat. Chem. Biol., 5:100-107 2009.
Clevers, Cell, 127:469-480, 2006.
Cole et al., Genes Dev., 22:746-755, 2008.
Coombs et al., J. Cell Sci.,123:3357-3367, 2010.
Ekker et al., Development, 116:1001-1010, 1992.
Fevr et al., Mol. Cell Biol., 27:7551-7559, 2007.
Galli et al., Development, 134:3339-3348, 2007.
Gao and Chen, Cell Signal, 22:717-727, 2010.
Gao et al., FEBS Lett., 585:2501-2506, 2011.
Greene and Wuts, In: Protective Groups in Organic Synthesis, 2^{nd} Ed.; Wiley, NY, 1999.
Handbook of Pharmaceutical Salts: Properties, Selection and Use, Stahl & Wermuth (Eds.), Verlag Helvetica Chimica Acta, 2002.
Herr and Basler, Dev. Biol., 361:392-402, 2012.
Huang and He, Curr. Opin. Cell Biol., 20(2):119-125, 2008.
Huang and He, Curr. Opin. Cell Biol., 20(2):119-125, 2008.
Huang et al., Nature, 461:614-620, 2009.
Jacob et al., Sci. Signal., 4(157):ra4, 2011.
Kinzler and Vogelstein, Cell, 87:159-170, 1996.
Korinek et al., Nat. Genet., 19:379-383, 1998.
Lee et al., Mol. Cell, 37:183-195, 2010.
Liu et al., Science, 317:803-806, 2007.
Lynch, Exp. Opin. Emerging Drugs, 9:345, 2004.
March's Advanced Organic Chemistry: Reactions, Mechanisms, and Structure (March's Advanced Organic Chemistry), Smith and March (Eds.), 2007.
Marlow et al., Development, 131:203-216, 2004.
Merkel et al., Pediatric Nephrol.(Germany), 22:1825-1838, 2007.
Mulligan et al., Proc. Natl. Acad. Sci. USA, 09(2):370-377, 2012
Muncan et al., EMBO Rpts., 8:966-973, 2007.
Polakis, Curr. Opin. Genet. Develop., 17:45-51, 2007.
Port and Basler, Traffic, 11:1265-1271, 2010.
Remington's Pharmaceutical Sciences, 18^{th} Ed. Mack Printing Company, 1289-1329, 1990.
Ren et al., J. Mol. Cell Cardiol., 51:280-287, 2011.
Reya and Clevers, Nature, 434:843-850, 2005.
Roszko et al., Sem. Cell Dev. Biol., 20:986-997, 2009.
Sato et al., Nature, 469:415-418, 2011.
Sepich et al., Development, 138:543-552, 2011.
Sjoblom et al., Science, 314:268-274, 2006.
Stoick-Cooper et al., Development, 134:479-489, 2007.
Takada et al., Dev. Cell, 11:791-801, 2006.
ten Berge et al., Nat. Cell Biol., 13:1070-1075, 2011.
van Amerongen and Nusse, Development, 136, 3205-3214, 2009.
Van der Flier et al., Gastroenterology, 132:628-632, 2007.
Veeman et al., Developmental Cell, 5:367, 2003.
Yang et al., Cell, 132:387-396, 2008.

1. A method of inhibiting Wnt protein signalling in a cell comprising administering to the cell an effective amount of a compound of the formula: 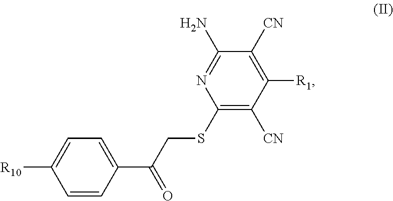
wherein:
R₁ is alkyl_{(C≦8)} or substituted alkyl_{(C≦8)}; and
R₁₀ is aryl_{(C≦8)}, substituted aryl_{(C≦8)}, heterocycloalkyl_{(C≦8)} or substituted heterocycloalkyl_{(C≦8)}; 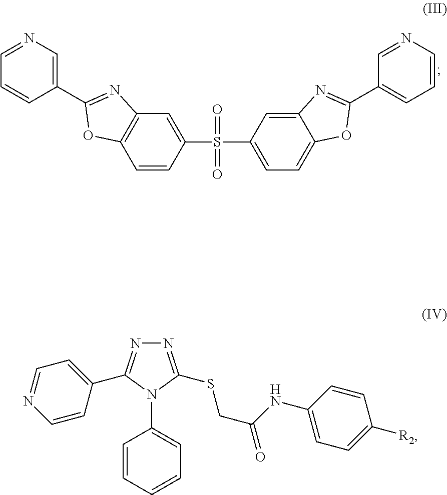
wherein R₂ is alkoxy_{(C≦8)}, substituted alkoxy_{(C≦8)}, acyl_{(C≦8)}, substituted acyl_{(C≦8)}, heterocycloalkyl_{(C≦8)} or substituted heterocycloalkyl_{(C≦8)}; 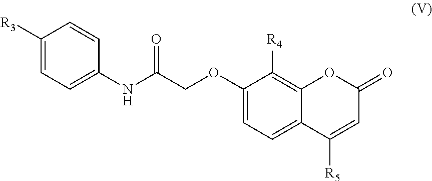
wherein:
R₃ is acyl_{(C≦8)}, substituted acyl_{(C≦8)}, heterocycloalkyl_{(C≦8)} or substituted heterocycloalkyl_{(C≦8)};
R₄ is hydrogen, alkyl_{(C≦8)} or substituted alkyl_{(C≦8)}; and
R₅ alkyl_{(C≦8)} or substituted alkyl_{(C≦8)}; 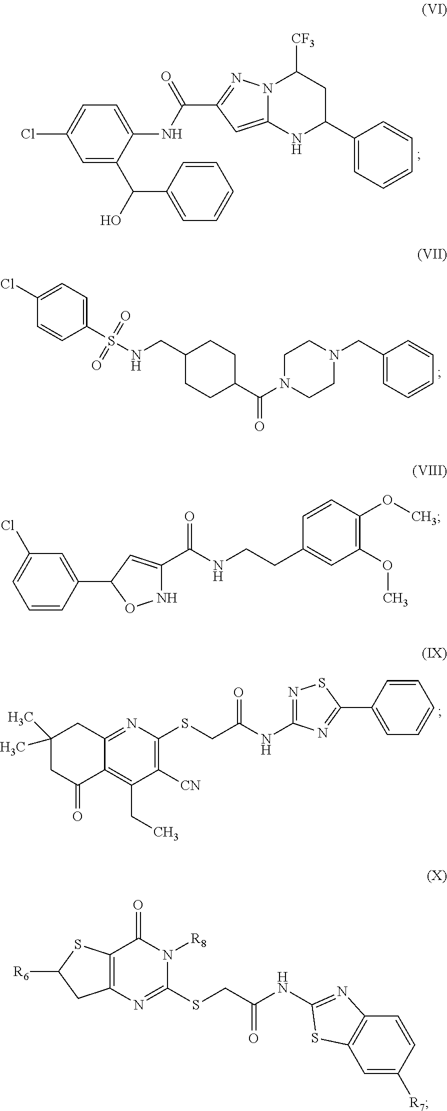
wherein:
R₆ is hydrogen, alkyl_{(C≦8)}, or substituted alkyl_{(C≦8)};
R₇ is hydrogen, alkyl_{(C≦8)} or substituted alkyl_{(C≦8)}; and
R₈ is alkyl_{(C≦8)}, substituted alkyl_{(C≦8)}, aryl_{(C≦8)}, or substituted aryl_{(C≦8)}; 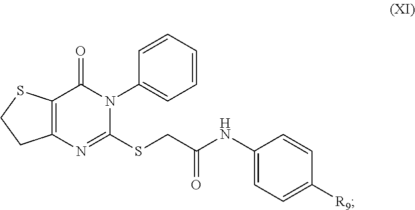
wherein:
R₉ is acyl_{(C≦8)}, substituted acyl_{(C≦8)}, alkyl_{(C≦8)}, substituted alkyl_{(C≦8)}, heterocycloalkyl_{(C≦8)} or substituted heterocycloalkyl_{(C≦8)}; or 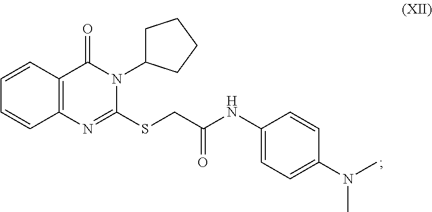 [SMILES: CN(C)c1ccc(NC(=O)CSc2nc3ccccc3c(=O)n2C2CCCC2)cc1]
or a pharmaceutically acceptable salt or tautomer thereof.

2. The method of claim 1, wherein the compound is further defined as a compound of formula II or a pharmaceutically acceptable salt or tautomer thereof.

3. The method of claim 2, wherein the compound is further defined as: 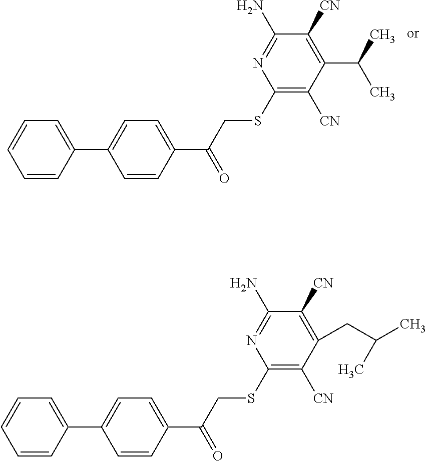
or a pharmaceutically acceptable salt or tautomer thereof.

4. The method of claim 1, wherein the compound is further defined as a compound of formula IV or a pharmaceutically acceptable salt or tautomer thereof.

5. The method of claim 4, wherein the compound is further defined as: 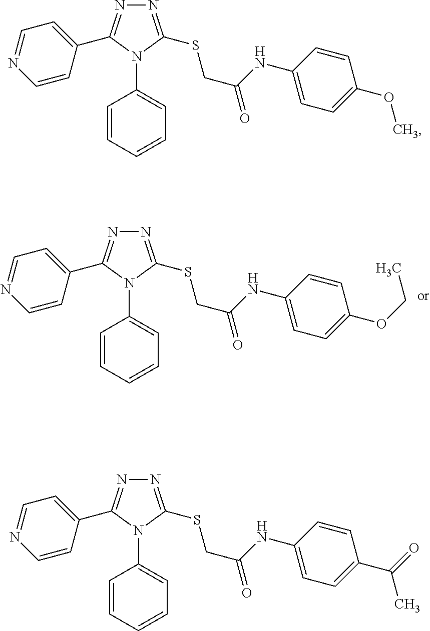 [SMILES: COc1ccc(NC(=O)CSc2nnc(-c3ccncc3)n2-c2ccccc2)cc1 ; CCOc1ccc(NC(=O)CSc2nnc(-c3ccncc3)n2-c2ccccc2)cc1 ; CC(=O)c1ccc(NC(=O)CSc2nnc(-c3ccncc3)n2-c2ccccc2)cc1]
or a pharmaceutically acceptable salt or tautomer thereof.

6. The method of claim 1, wherein the compound is further defined as a compound of formula V or a pharmaceutically acceptable salt or tautomer thereof.

7. The method of claim 6, wherein the compound is further defined as: 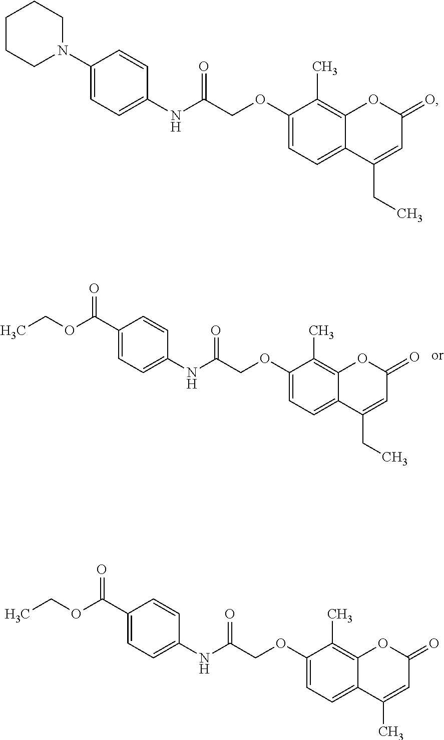 [SMILES: CCc1cc(=O)oc2c(C)c(OCC(=O)Nc3ccc(N4CCCCC4)cc3)ccc12 ; CCOC(=O)c1ccc(NC(=O)COc2ccc3c(CC)cc(=O)oc3c2C)cc1 ; "or" ; CCOC(=O)c1ccc(NC(=O)COc2ccc3c(C)cc(=O)oc3c2C)cc1]
or a pharmaceutically acceptable salt or tautomer thereof.

8. The method of claim 1, wherein the compound is further defined as a compound of formula X or a pharmaceutically acceptable salt or tautomer thereof.

9. The method of claim 8, wherein the compound is further defined as: 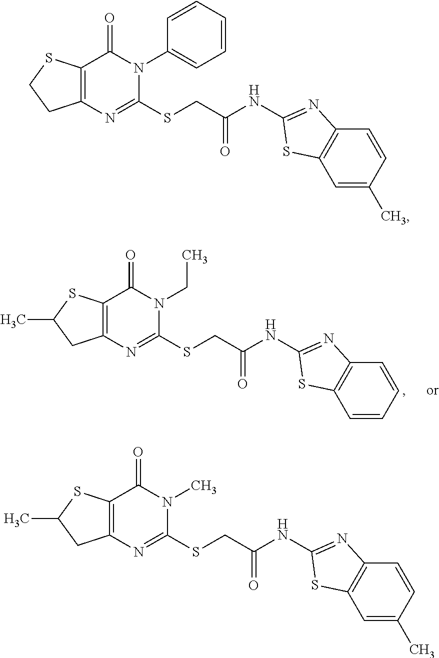
or a pharmaceutically acceptable salt or tautomer thereof.

10. The method of claim 1, wherein the compound is further defined as a compound of formula XI or a pharmaceutically acceptable salt or tautomer thereof.

11. The method of claim 10, wherein the compound is further defined as: 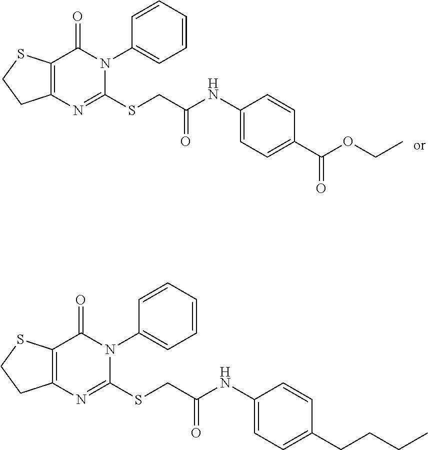 [SMILES: CCOC(=O)c1ccc(NC(=O)CSc2nc3c(c(=O)n2-c2ccccc2)SCC3)cc1 ; CCCCc1ccc(NC(=O)CSc2nc3c(c(=O)n2-c2ccccc2)SCC3)cc1]
or a pharmaceutically acceptable salt or tautomer thereof.

12. The method of claim 1, wherein the compound is selected from formulas III, VI-IX and XII or a pharmaceutically acceptable salt or tautomer thereof.

13. The method of claim 1, wherein the administering is performed in vitro.

14. The method of claim 1, wherein the administering is performed in vivo.

15. The method of claim 1, wherein the method of inhibiting Wnt protein signalling is further defined as a method of inhibiting Wnt response.

16. The method of claim 1, wherein the method of inhibiting Wnt protein signalling is further defined as a method of inhibiting Wnt protein production.

17. The method of claim 1, further comprising administering to said cell an inhibitor of a Tankyrase enzymes and/or a inhihitor of GSK-3β.

18. A method of treating cancer in a subject comprising administering to the subject an effective amount of a compound of the formula: 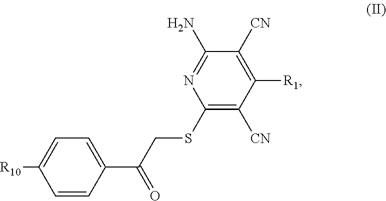
wherein:
R₁ is alkyl_{(C≦8)} or substituted alkyl_{(C≦8)}; and
R₁₀ is aryl_{(C≦8)}, substituted aryl_{(C≦8)}, heterocycloalkyl_{(C≦8)} or substituted heterocycloalkyl_{(C≦8)}; 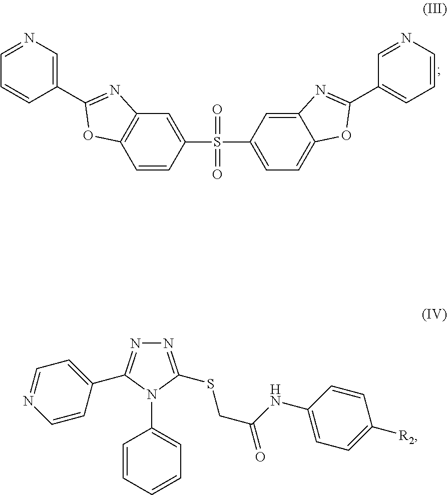
wherein R₂ is alkoxy_{(C≦8)}, substituted alkoxy_{(C≦8)}, acyl_{(C≦8)}, substituted acyl_{(C≦8)}, heterocycloalkyl_{(C≦8)} or substituted heterocycloalkyl_{(C≦8)}; 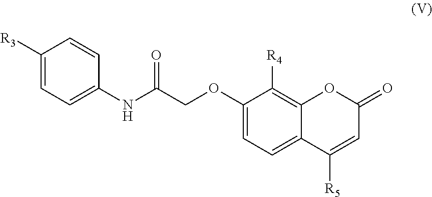
wherein:
R₃ is acyl_{(C≦8)}, substituted acyl_{(C≦8)}, heterocycloalkyl_{(C≦8)} or substituted heterocycloalkyl_{(C≦8)};
R₄ is hydrogen, alkyl_{(C≦8)} or substituted alkyl_{(C≦8)}; and
R₅ alkyl_{(C≦8)} or substituted alkyl_{(C≦8)}; 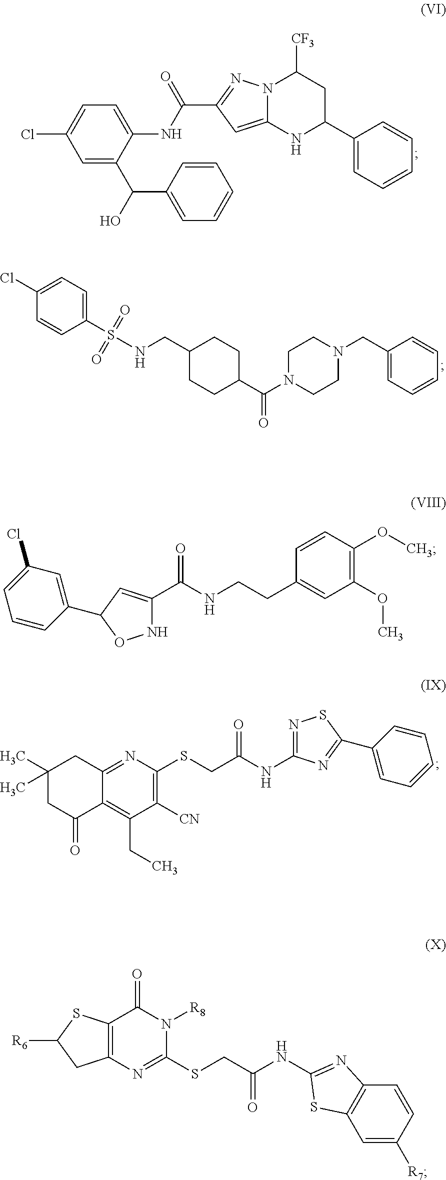
wherein:
R₆ is hydrogen, alkyl_{(C≦8)}, or substituted alkyl_{(C≦8)};
R₇ is hydrogen, alkyl_{(C≦8)} or substituted alkyl_{(C≦8)}; and
R₈ is alkyl_{(C≦8)}, substituted alkyl_{(C≦8)}, aryl_{(C≦8)}, or substituted aryl_{(C≦8)}; 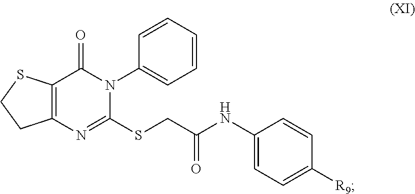
wherein:
R₉ is acyl_{(C≦8)}, substituted acyl_{(C≦8)}, alkyl_{(C≦8)}, substituted alkyl_{(C≦8)}, heterocycloalkyl_{(C≦8)} or substituted heterocycloalkyl_{(C≦8)}; or 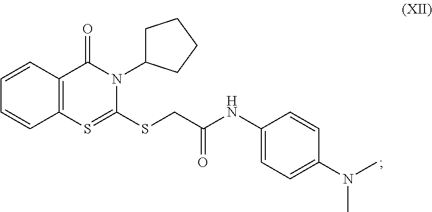
or a pharmaceutically acceptable salt or tautomer thereof.

19-34. (canceled)

35. A method of treating or preventing osteopetrosis in a patient comprising administering to the patient an effective amount of a compound of the formula: 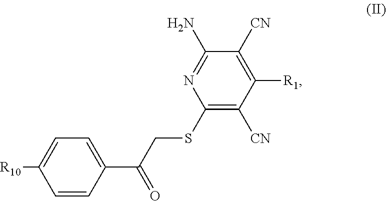
wherein:
R₁ is alkyl_{(C≦8)} or substituted alkyl_{(C≦8)}; and
R₁₀ is aryl_{(C≦8)}, substituted aryl_{(C≦8)}, heterocycloalkyl_{(C≦8)} or substituted heterocycloalkyl_{(C≦8)}; 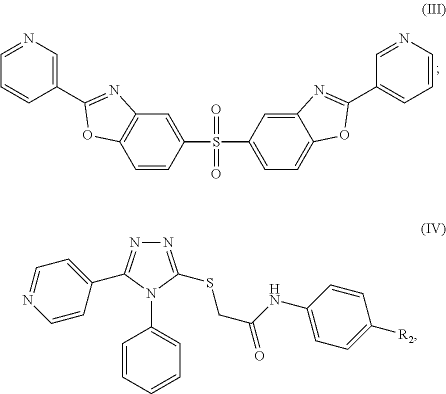
wherein R₂ is alkoxy_{(C≦8)}, substituted alkoxy_{(C≦8)}, acyl_{(C≦8)}, substituted acyl_{(C≦8)}, heterocycloalkyl_{(C≦8)} or substituted heterocycloalkyl_{(C≦8)}; 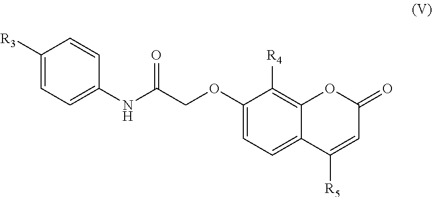
wherein:
R₃ is acyl_{(C≦8)}, substituted acyl_{(C≦8)}, heterocycloalkyl_{(C≦8)} or substituted heterocycloalkyl_{(C≦8)};
R₄ is hydrogen, alkyl_{(C≦8)} or substituted alkyl_{(C≦8)}; and
R₅ alkyl_{(C≦8)} or substituted alkyl_{(C≦8)}; 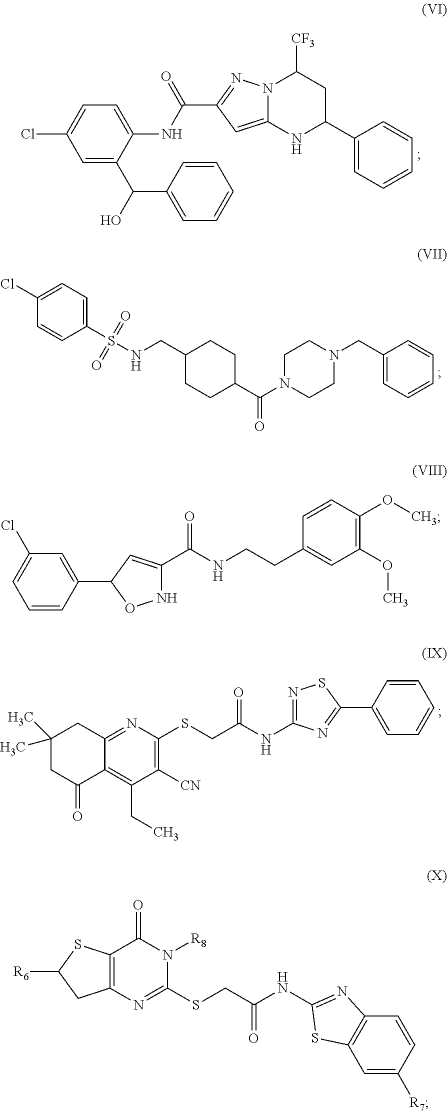
wherein:
R₆ is hydrogen, alkyl_{(C≦8)}, or substituted alkyl_{(C≦8)};
R₇ is hydrogen, alkyl_{(C≦8)} or substituted alkyl_{(C≦8)}; and
R₈ is alkyl_{(C≦8)}, substituted alkyl_{(C≦8)}, aryl_{(C≦8)}, or substituted aryl_{(C≦8)}; 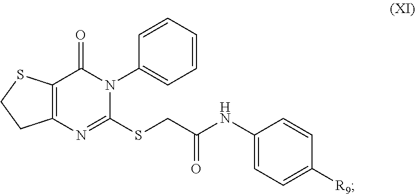
wherein:
R₉ is acyl_{(C≦8)}, substituted acyl_{(C≦8)}, alkyl_{(C≦8)}, substituted alkyl_{(C≦8)}, heterocycloalkyl_{(C≦8)} or substituted heterocycloalkyl_{(C≦8)}; or 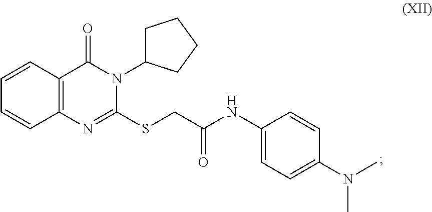 [SMILES: CN(C)c1ccc(NC(=O)CSc2nc3ccccc3c(=O)n2C2CCCC2)cc1]
or a pharmaceutically acceptable salt or tautomer thereof.

36-51. (canceled)

52. A method of treating a degenerative disease in a patient comprising administering to the patient an effective amount of a compound of the formula: 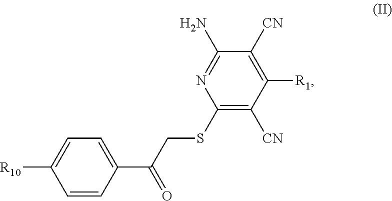
wherein:
R₁ is alkyl_{(C≦8)} or substituted alkyl_{(C≦8)}; and
R₁₀ is aryl_{(C≦8)}, substituted aryl_{(C≦8)}, heterocycloalkyl_{(C≦8)} or substituted heterocycloalkyl_{(C≦8)}; 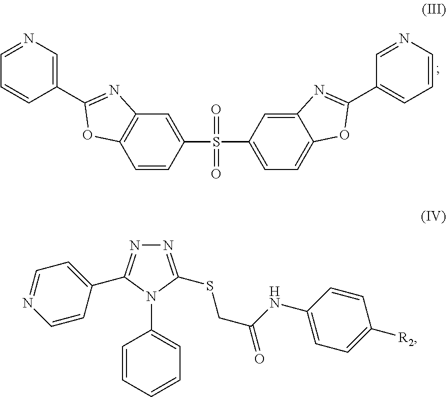
wherein R₂ is alkoxy_{(C≦8)}, substituted alkoxy_{(C≦8)}, acyl_{(C≦8)}, substituted acyl_{(C≦8)}, heterocycloalkyl_{(C≦8)} or substituted heterocycloalkyl_{(C≦8)}; 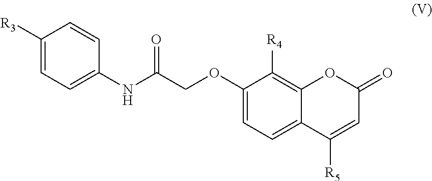
wherein:
R₃ is acyl_{(C≦8)}, substituted acyl_{(C≦8)}, heterocycloalkyl_{(C≦8)} or substituted heterocycloalkyl_{(C≦8)};
R₄ is hydrogen, alkyl_{(C≦8)} or substituted alkyl_{(C≦8)}; and
R₅ alkyl_{(C≦8)} or substituted alkyl_{(C≦8)}; 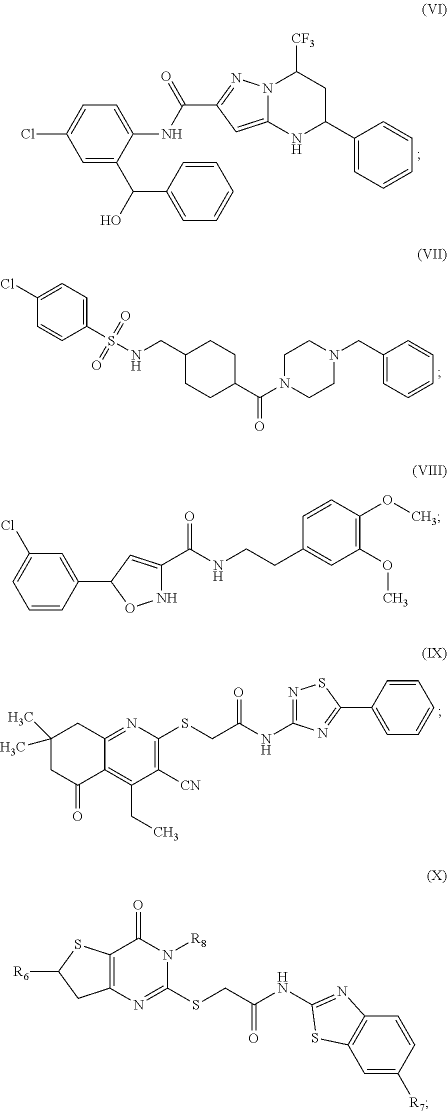
wherein:
R₆ is hydrogen, alkyl_{(C≦8)}, or substituted alkyl_{(C≦8)};
R₇ is hydrogen, alkyl_{(C≦8)} or substituted alkyl_{(C≦8)}; and
R₈ is alkyl_{(C≦8)}, substituted alkyl_{(C≦8)}, aryl_{(C≦8)}, or substituted aryl_{(C≦8)}; 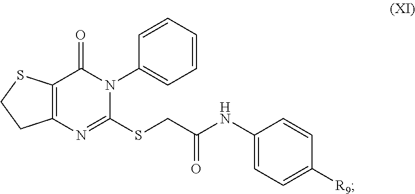
wherein:
R₉ is acyl_{(C≦8)}, substituted acyl_{(C≦8)}, alkyl_{(C≦8)}, substituted alkyl_{(C≦8)}, heterocycloalkyl_{(C≦8)} or substituted heterocycloalkyl_{(C≦8)}; or 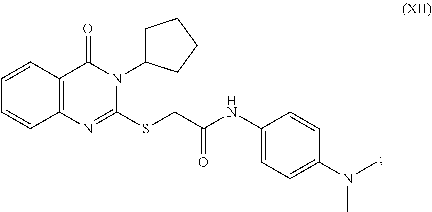 [SMILES: CN(C)c1ccc(NC(=O)CSc2nc3ccccc3c(=O)n2C2CCCC2)cc1]
or a pharmaceutically acceptable salt or tautomer thereof.

53-68. (canceled)

69. A pharmaceutical composition comprising a pharmaceutically acceptable carrier, diluent, and/or excipient and a compound of the formula: 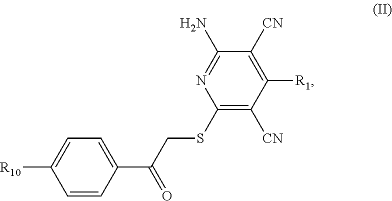
wherein:
R₁ is alkyl_{(C≦8)} or substituted alkyl_{(C≦8)}; and
R₁₀ is aryl_{(C≦8)}, substituted aryl_{(C≦8)}, heterocycloalkyl_{(C≦8)} or substituted heterocycloalkyl_{(C≦8)}; 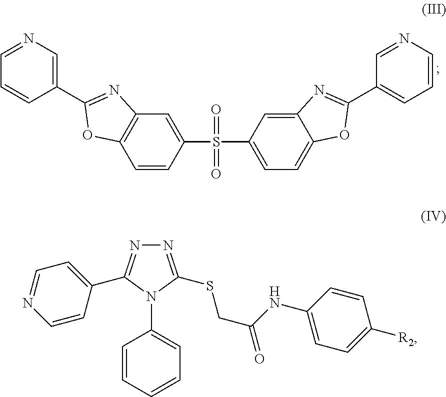
wherein R₂ is alkoxy_{(C≦8)}, substituted alkoxy_{(C≦8)}, acyl_{(C≦8)}, substituted acyl_{(C≦8)}, heterocycloalkyl_{(C≦8)} or substituted heterocycloalkyl_{(C≦8)}; 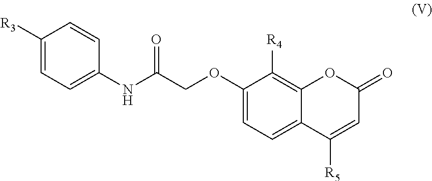
wherein:
R₃ is acyl_{(C≦8)}, substituted acyl_{(C≦8)}, heterocycloalkyl_{(C≦8)} or substituted heterocycloalkyl_{(C≦8)};
R₄ is hydrogen, alkyl_{(C≦8)} or substituted alkyl_{(C≦8)}; and
R₅ alkyl_{(C≦8)} or substituted alkyl_{(C≦8)}; 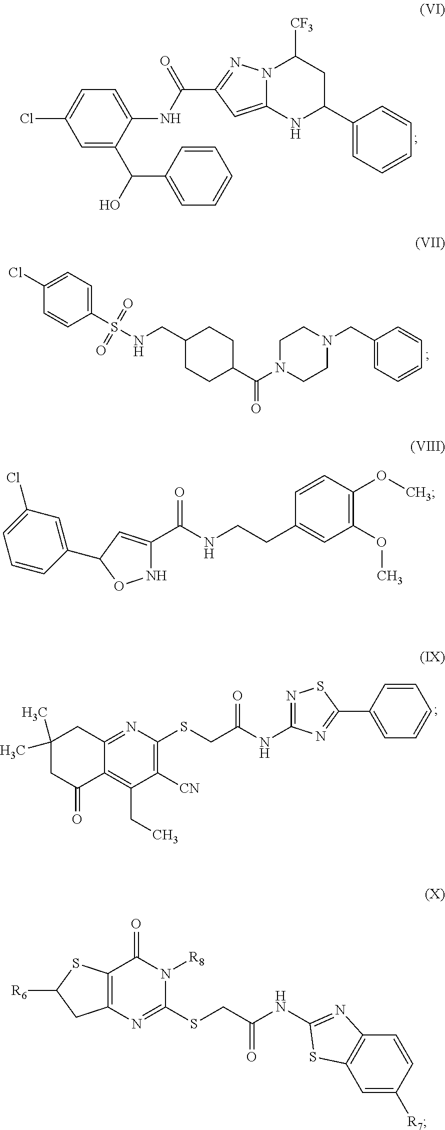
wherein:
R₆ is hydrogen, alkyl_{(C≦8)}, or substituted alkyl_{(C≦8)};
R₇ is hydrogen, alkyl_{(C≦8)} or substituted alkyl_{(C≦8)}; and
R₈ is alkyl_{(C≦8)}, substituted alkyl_{(C≦8)}, aryl_{(C≦8)}, or substituted aryl_{(C≦8)}; 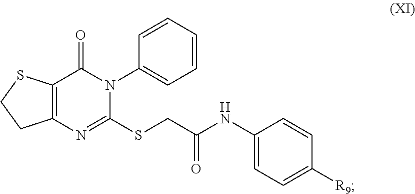
wherein:
R₉ is acyl_{(C≦8)}, substituted acyl_{(C≦8)}, alkyl_{(C≦8)}, substituted alkyl_{(C≦8)}, heterocycloalkyl_{(C≦8)} or substituted heterocycloalkyl_{(C≦8)}; or 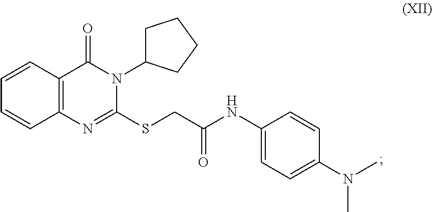 [SMILES: CN(C)c1ccc(NC(=O)CSc2nc3ccccc3c(=O)n2C2CCCC2)cc1]
or a pharmaceutically acceptable salt or tautomer thereof.

70-80. (canceled)
